SEQUENCE LISTING (36 sequences)

SEQ ID NO: 1 - DNA, 1339 bp, Homo sapiens
  misc_feature: Gene ID ANXA3
gaattccgattagtgtgatctcagctcaaggcaaaggtgggatatcatggcatctatctgggttggacaccgaggaacagtaagagattatccagactttagcccatcagtggatgctgaagctattcagaaagcaatcagaggaattggaactgatgagaaaatgctcatcagcattctgactgagaggtcaaatgcacagcggcagctgattgttaaggaatatcaagcagcatatggaaaggagctgaaagatgacttgaagggtgatctctctggccactttgagcatctcatggtggccctagtgactccaccagcagtctttgatgcaaagcagctaaagaaatccatgaagggcgcgggaacaaacgaagatgccttgattgaaatcttaactaccaggacaagcaggcaaatgaaggatatctctcaagcctattatacagtatacaagaagagtcttggagatgacattagttccgaaacatctggtgacttccggaaagctctgttgactttggcagatggcagaagagatgaaagtctgaaagtggatgagcatctggccaaacaagatgcccagattctctataaagctggtgagaacagatggggcacggatgaagacaaattcactgagatcctgtgtttaaggagctttcctcaattaaaactaacatttgatgaatacagaaatatcagccaaaaggacattgtggacagcataaaaggagaattatctgggcattttgaagacttactgttggccatagttaattgtgtgaggaacacgccggcctttttagccgaaagactgcatcgagccttgaagggtattggaactgatgagtttactctgaaccgaataatggtgtccagatcagaaattgaccttttggacattcgaacagagttcaagaagcattatggctattccctatattcagcaattaaatcggatacttctggagactatgaaatcacactcttaaaaatctgtggtggagatgactgaaccaagaagataatctccaaaggtccacgatgggctttcccaacagctccaccttacttcttctcatactatttaagagaacaagcaaatataaacagcaacttgtgttcctaacaggaattttcattgttctataacaacaacaacaaaagcgattattattttagagcatctcatttataatgtagcagctcataaatgaaattgaaaatggtattaaagatctgcaactactatccaacttatatttctgctttcaaagttaagaatctttatagttctactccattaaatataaagcaagataataaaacggaattc

SEQ ID NO: 2 - DNA, 1749 bp, Homo sapiens
  misc_feature: Gene ID  AP1M2
ggcgcttccgcaggaagaaggaagcggcgccgccatcgcctcccggcgctccctccccgactcctaagtccttcggccgccaccatgtccgcctcggctgtcttcattctggacgttaagggcaagccattgatcagccgcaactacaagggcgatgtggccatgagcaagattgagcacttcatgcctttgctggtacagcgggaggaggaaggcgccctggccccgctgctgagccacggccaggtccacttcctatggatcaaacacagcaacctctacttggtggccaccacatcgaagaatgccaatgcctccctggtgtactccttcctgtataagacaatagaggtattctgcgaatacttcaaggagctggaggaggagagcatccgggacaactttgtcatcgtctacgagttgctggacgagctcatggactttggcttcccgcagaccaccgacagcaagatcctgcaggagtacatcactcagcagagcaacaagctggagacgggcaagtcacgggtgccacccactgtcaccaacgctgtgtcctggcgctccgagggtatcaagtataagaagaacgaggtcttcattgatgtcatagagtctgtcaacctgctggtcaatgccaacggcagcgtccttctgagcgaaatcgtcggtaccatcaagctcaaggtgtttctgtcaggaatgccagagctgcggctgggcctcaatgaccgcgtgctcttcgagctcactggccgcagcaagaacaaatcagtagagctggaggatgtaaaattccaccagtgcgtgcggctctctcgctttgacaacgaccgcaccatctccttcatcccgcctgatggtgactttgagctcatgtcataccgcctcagcacccaggtcaagccactgatctggattgagtctgtcattgagaagttctcccacagccgcgtggagatcatggtcaaggccaaggggcagtttaagaaacagtcagtggccaacggtgtggagatatctgtgcctgtacccagcgatgccgactcccccagattcaagaccagtgtgggcagcgccaagtatgtgccggagagaaacgtcgtgatttggagtattaagtctttcccggggggcaaggagtacttgatgcgagcccactttggcctccccagtgtggaaaaggaagaggtggagggccggccccccatcggggtcaagtttgagatcccctacttcaccgtctctgggatccaggtccgatacatgaagatcattgagaaaagtggttaccaggccctgccctgggttcgctacatcacccagagtggcgattaccaacttcgtaccagctagaagggagaagagatgggggcttgaacacggggcttccttacagccccggatgcagattttagagggagggcaggtgcgggctgtgtgtgtctgtgtgagggcaggtcctggacttggcagtttcttgctcccagcacccgccccttcctcacctcttccttattccataggctgggagagaaactctctgcttccctcgcccttggagctttccccatccccctgattttatatgaagaaatagaagaggggcttgaagtccccctcgcgagtgccttcttgcaattacctgccttagcgggtgttgcgggtccctccttcacagccgctgagcccagaggtcccgctggcccctcctctgaattttaggatgtcattaaaaagatgaatcta

SEQ ID NO: 3 - DNA, 1568 bp, Homo sapiens
  misc_feature: Gene ID ATP1B1
cagcggcgcgtcctgcctgcagagagccaggccggagaagccgagcggcgcagaggacgccagggcgcgcgccgcagccacccaccctccggaccgcggcagctgctgacccgccatcgccatggcccgcgggaaagccaaggaggagggcagctggaagaaattcatctggaactcagagaagaaggagtttctgggcaggaccggtggcagttggtttaagatccttctattctacgtaatattttatggctgcctggctggcatcttcatcggaaccatccaagtgatgctgctcaccatcagtgaatttaagcccacatatcaggaccgagtggccccgccaggattaacacagattcctcagatccagaagactgaaatttcctttcgtcctaatgatcccaagagctatgaggcatatgtactgaacatagttaggttcctggaaaagtacaaagattcagcccagagggatgacatgatttttgaagattgtggcgatgtgcccagtgaaccgaaagaacgaggagactttaatcatgaacgaggagagcgaaaggtctgcagattcaagcttgaatggctgggaaattgctctggattaaatgatgaaacttatggctacaaagagggcaaaccgtgcattattataaagctcaaccgagttctaggcttcaaacctaagcctcccaagaatgagtccttggagacttacccagtgatgaagtataacccaaatgtccttcccgttcagtgcactggcaagcgagatgaagataaggataaagttggaaatgtggagtattttggactgggcaactcccctggttttcctctgcagtattatccgtactatggcaaactcctgcagcccaaatacctgcagcccctgctggccgtacagttcaccaatcttaccatggacactgaaattcgcatagagtgtaaggcgtacggtgagaacattgggtacagtgagaaagaccgttttcagggacgttttgatgtaaaaattaaattttaagtgacactacagaaaaacacaaaaaggtgatgggttgtgttatgcttgtattgaatgctgtcttgacatctcttgccttgtcctccggtatgttctaaagctgtgtctgagatctggatctgcccatcactttggctagtgacagggctaattaatttgctttatacattttcttttactttccttttttcctttctggaggcatcacatgctggtgctgtgtctttatgaatgttttaaccattttcatggtggaagaattttatatttatgcagttgtacaattttatttttttctgcaagaaaaagtgtaatgtatgaaataaaccaaagtcacttgtttgaaaataaatctttattttgaactttataaaaagcaatgcagtaccccatagactggtgttaaatgttgtctacagtgcaaaatccatgttctaacatatgtaataattgccaggagtacagtgctcttgttgatcttgtattcagtcaggttaaaacaacggacaataaaagaatgaacacattcaaaaaaaaaaaa

SEQ ID NO: 4 - DNA, 5511 bp, Homo sapiens
  misc_feature: Gene ID C11orf9
acacacacacacccctcccctgccatccctccccggactccggctccggctccgattgcaatttgcaacctccgctgccgtcgccgcagcagccaccaattcgccagcggttcaggtggctcttgcctcgatgtcctagcctaggggcccccgggccggacttggctgggctcccttcaccctctgcggagtcatgagggcgaacgacgctctgcaggtgctgggcttgcttttcagcctggcccggggctccgaggtgggcaactctcaggcagtgtgtcctgggactctgaatggcctgagtgtgaccggcgatgctgagaaccaataccagacactgtacaagctctacgagaggtgtgaggtggtgatggggaaccttgagattgtgctcacgggacacaatgccgacctctccttcctgcagtggattcgagaagtgacaggctatgtcctcgtggccatgaatgaattctctactctaccattgcccaacctccgcgtggtgcgagggacccaggtctacgatgggaagtttgccatcttcgtcatgttgaactataacaccaactccagccacgctctgcgccagctccgcttgactcagctcaccgagattctgtcagggggtgtttatattgagaagaacgataagctttgtcacatggacacaattgactggagggacatcgtgagggaccgagatgctgagatagtggtgaaggacaatggcagaagctgtcccccctgtcatgaggtttgcaaggggcgatgctggggtcctggatcagaagactgccagacattgaccaagaccatctgtgctcctcagtgtaatggtcactgctttgggcccaaccccaaccagtgctgccatgatgagtgtgccgggggctgctcaggccctcaggacacagactgctttgcctgccggcacttcaatgacagtggagcctgtgtacctcgctgtccacagcctcttgtctacaacaagctaactttccagctggaacccaatccccacaccaagtatcagtatggaggagtttgtgtagccagctgtccccataactttgtggtggatcaaacatcctgtgtcagggcctgtcctcctgacaagatggaagtagataaaaatgggctcaagatgtgtgagccttgtgggggactatgtcccaaagcctgtgagggaacaggctctgggagccgcttccagactgtggactcgagcaacattgatggatttgtgaactgcaccaagatcctgggcaacctggactttctgatcaccggcctcaatggagacccctggcacaagatccctgccctggacccagagaagctcaatgtcttccggacagtacgggagatcacaggttacctgaacatccagtcctggccgccccacatgcacaacttcagtgttttttccaatttgacaaccattggaggcagaagcctctacaaccggggcttctcattgttgatcatgaagaacttgaatgtcacatctctgggcttccgatccctgaaggaaattagtgctgggcgtatctatataagtgccaataggcagctctgctaccaccactctttgaactggaccaaggtgcttcgggggcctacggaagagcgactagacatcaagcataatcggccgcgcagagactgcgtggcagagggcaaagtgtgtgacccactgtgctcctctgggggatgctggggcccaggccctggtcagtgcttgtcctgtcgaaattatagccgaggaggtgtctgtgtgacccactgcaactttctgaatggggagcctcgagaatttgcccatgaggccgaatgcttctcctgccacccggaatgccaacccatggagggcactgccacatgcaatggctcgggctctgatacttgtgctcaatgtgcccattttcgagatgggccccactgtgtgagcagctgcccccatggagtcctaggtgccaagggcccaatctacaagtacccagatgttcagaatgaatgtcggccctgccatgagaactgcacccaggggtgtaaaggaccagagcttcaagactgtttaggacaaacactggtgctgatcggcaaaacccatctgacaatggctttgacagtgatagcaggattggtagtgattttcatgatgctgggcggcacttttctctactggcgtgggcgccggattcagaataaaagggctatgaggcgatacttggaacggggtgagagcatagagcctctggaccccagtgagaaggctaacaaagtcttggccagaatcttcaaagagacagagctaaggaagcttaaagtgcttggctcgggtgtctttggaactgtgcacaaaggagtgtggatccctgagggtgaatcaatcaagattccagtctgcattaaagtcattgaggacaagagtggacggcagagttttcaagctgtgacagatcatatgctggccattggcagcctggaccatgcccacattgtaaggctgctgggactatgcccagggtcatctctgcagcttgtcactcaatatttgcctctgggttctctgctggatcatgtgagacaacaccggggggcactggggccacagctgctgctcaactggggagtacaaattgccaagggaatgtactaccttgaggaacatggtatggtgcatagaaacctggctgcccgaaacgtgctactcaagtcacccagtcaggttcaggtggcagattttggtgtggctgacctgctgcctcctgatgataagcagctgctatacagtgaggccaagactccaattaagtggatggcccttgagagtatccactttgggaaatacacacaccagagtgatgtctggagctatggtgtgacagtttgggagttgatgaccttcggggcagagccctatgcagggctacgattggctgaagtaccagacctgctagagaagggggagcggttggcacagccccagatctgcacaattgatgtctacatggtgatggtcaagtgttggatgattgatgagaacattcgcccaacctttaaagaactagccaatgagttcaccaggatggcccgagacccaccacggtatctggtcataaagagagagagtgggcctggaatagcccctgggccagagccccatggtctgacaaacaagaagctagaggaagtagagctggagccagaactagacctagacctagacttggaagcagaggaggacaacctggcaaccaccacactgggctccgccctcagcctaccagttggaacacttaatcggccacgtgggagccagagccttttaagtccatcatctggatacatgcccatgaaccagggtaatcttggggagtcttgccaggagtctgcagtttctgggagcagtgaacggtgcccccgtccagtctctctacacccaatgccacggggatgcctggcatcagagtcatcagaggggcatgtaacaggctctgaggctgagctccaggagaaagtgtcaatgtgtaggagccggagcaggagccggagcccacggccacgcggagatagcgcctaccattcccagcgccacagtctgctgactcctgttaccccactctccccacccgggttagaggaagaggatgtcaacggttatgtcatgccagatacacacctcaaaggtactccctcctcccgggaaggcaccctttcttcagtgggtctcagttctgtcctgggtactgaagaagaagatgaagatgaggagtatgaatacatgaaccggaggagaaggcacagtccacctcatccccctaggccaagttcccttgaggagctgggttatgagtacatggatgtggggtcagacctcagtgcctctctgggcagcacacagagttgcccactccaccctgtacccatcatgcccactgcaggcacaactccagatgaagactatgaatatatgaatcggcaacgagatggaggtggtcctgggggtgattatgcagccatgggggcctgcccagcatctgagcaagggtatgaagagatgagagcttttcaggggcctggacatcaggccccccatgtccattatgcccgcctaaaaactctacgtagcttagaggctacagactctgcctttgataaccctgattactggcatagcaggcttttccccaaggctaatgcccagagaacgtaactcctgctccctgtggcactcagggagcatttaatggcagctagtgcctttagagggtaccgtcttctccctattccctctctctcccaggtcccagccccttttccccagtcccagacaattccattcaatctttggaggcttttaaacattttgacacaaaattcttatggtatgtagccagctgtgcactttcttctctttcccaaccccaggaaaggttttccttattttgtgtgctttcccagtcccattcctcagcttcttcacaggcactcctggagatatgaaggattactctccatatcccttcctctcaggctcttgactacttggaactaggctcttatgtgtgcctttgtttcccatcagactgtcaagaagaggaaagggaggaaacctagcagaggaaagtgtaattttggtttatgactcttaaccccctagaaagacagaagcttaaaatctgtgaagaaagaggttaggagtagatattgattactatcataattcagcacttaactatgagccaggcatcatactaaacttcacctacattatctcacttagtcctttatcatccttaaaacaattctgtgacatacatattatctcattttacacaaagggaagtcgggcatggtggctcatgcctgtaatctcagcactttgggaggctgaggcagaaggattacctgaggcaaggagtttgagaccagcttagccaacatagtaagacccccatctctttaaaaaaaaaaaaaaaaaaaaaaaaaaaactttagaactgggtgcagtggctcatgcctgtaatcccagccagcactttgggaggctgagatgggaagatcacttgagcccagaattagagataagcctatggaaacatagcaagacactgtctctacaggggaaaaaaaaaaaagaaactgagccttaaagagatgaaataaattaagcagtagatccaggatgcaaaatcctcccaattcctgtgcatgtgctcttattgtaaggtgccaagaaaaactgatttaagttacagcccttgtttaaggggcactgtttcttgtttttgcactgaatcaagtctaaccccaacagccacatcctcctatacctagacatctcatctcaggaagtggtggtgggggtagtcagaaggaaaaataactggacatctttgtgtaaaccataatccacatgtgccgtaaatgatcttcactccttatccgagggcaaattcacaaggatccccaagatccacttttagaagccattctcatcca

SEQ ID NO: 5 - DNA, 4098 bp, Homo sapiens
  misc_feature: Gene ID  C1orf106
atgctgcaaatgccgaagttaaatgaaatacctccggggagggcaggccgcagggaggctcggggggagggaagatggcctggacaaacaggtcctgaagctgcgaggctggagtggagggcgcaggggcaggcgggcggcgccagagctccatgggacagctggggaagctccaggctacctacacaacctggcccaggctggtcacggtgtcccccctccctgctctgtgccctctccttccagaaatccaccatggagagtaaggatgaggtcagcgacaccgacagtggcatcatcctgcagtctggccccgacagcccggtctccccaatgaaggagctgacccatgcagtgcacaagcagcagagggccctggaagcgaggctggaggcctgcctggaggagctgaggagactctgccttcgggaagcggagctgacgggcaccttgccagcggagtatcccctcaaaccaggggaaaaggcccccaaggttcgccgcaggatcggagcggcttacaaactggatgactgggccttgcacagagaggaccccctaagcagcctggagcgccagctggccctgcagctgcagatcacagaggcagcccgtcggctgtgcctggaggagaacctcagcaggcaggctcggcggcagcggaagcactccatgctgcaggaggagaagaagctgcaggagctccagcgctgcctggtcgagcggcggcgcaatagcgagccacctccggctgctgctctccccctgggccgagagctcagtgcctctgatgacagctccctgtcagatgggctcctcctggaggaagaggaatcccaagtgccaaaacctcctccagagtctccagccccaccttctcggcctctcccaccccaaacccttgagggtctgcagccaacaggacctgaggctgggagcccagaacgggctccagtccagaacagcccctggaaggaaaccagcctggaccacccctatgagaagcccaggaagtcttctgagccctggagcgagtccagcagcccagccaccacaccacaggatgggcccagtgcctccagcctgtggcttctggagcctgcctcctaccacgtggttcccatccgtggtgttcctggccagtggcagggccgcaccagtgccccagccacccctgagatacaggggaggaggggccagtcgcagtctctgagggtggattccttccgggcgggtcctgagggccgaggtcgcagcgcctttccccgccgccgccccactcactacacggtgacagtgccagattcctgctttcccgcgaccaagcccccgctgccccacgccgcctgccactcctgctcagaagacagtggctctgacgtctccagcatctcccaccccacttcgccgggcagcagcagccccgacatctcctttctgcagcctctctcccctcccaagacccatcgtcaccgcggggcctgggtcccagccggcagcagagagctggtcgcccaccaccccaagctactgctgccgcctggctatttcccggcggggcggtacgtggtggtggctgagagccccctgccgcctggcgagtgggagctgtgccgcgcagccccgggccctgcttacgaggaggagggcactcccctgcgctaccagcgtctggtgccctcccgcagccgcatcgtgcggacgccctccctgaaggacagcccggcaggccgggggctcagcaaggccgccgtgtccgaggagctcaagtggtggcacgagcgtgcacgcctccggagcacccgaccccactcactggaccgccaaggagctttccgggtcaggagcctgccccttgggagagagggcttcggacgagccctgggaccccgggcacaggtgcccacagtttgtgtgctgtggagatcgcctgatggggcccctgtgcaagtctttgtacctgaaaaaggagagatcatcagccaggtgtaactctgcgccccacgctggaaaaaactgtttcatagaggggctgggctgagacccccccacccctgagtgcctctttcagctcccccatccccatcgcaggccgatgacctggagctaagaccttttattattttttttttacacgacttttttcagaagccctgacctaaggatttatatatgtggattgtcctcaatacccctgtgatatgattatgttttatcccccagagtttggcctactggacttaaggccttgcctgtctgactgacagcctctatctccttatataagacaagtggcaggggacgagtgaagcagagtgagccaccttgggagttctccaacgctctgtgctctggttctaagaaattccctggggaactgcccctggccctcctgtcccactattgctggaggctggacatggtacatactcatgcacatgactctcccccatttcccaggtctctgggtaccccagcctgggctgggggagaatctcttcccccttttctaatgtgctctgtgatgcacacaccaagtggtaggtcaaaggtcagtatatcccggtggtgtattgtcttgctagaccctgctattttcctgaccccctaaatcctctttagggacccagtcactataccctgtctatgccctgtgggctcccagacccctgagctttgagtcagtggcatcacagtttgtagcctcagggggtctggctgggggctggtccatgcttgtggttagtggacagcagccaccctttgacagctacctctggacatctcaagggcttgcagccccactgctccttctaacattttgtttgtttttgagatggagtctcgctctgtcgcccaggctggaatgctgtagcaagatttcggctcactgcaacccccgtctcccgggttcaagcgaatctcctgcctcagccttctgagtagctgggattacaggcaagcaccaccatgcctgctaattttttatttttagtagagatggggtttcatcatgttggccaggctggtctcgaactcctgacctcaagtgatccacctgccttggcctttcaaagtgctgggattacaggcatgagccaccgcacctggcccttctaacgttttttcatcatagtcccaaaaaccaatactttacaagtggtttttgaaaggcaccacttttgtggcatgttctggttgggagagggagtcacagttcctactccccccaccagctatgcttctgctctgagaaggtggttatttatacaaacatggacatactcactcccaagggctgatgagatgctgaattttctttgggggcattcattaattgtcccagctgcagcgactggagcaagtctggaagctgcctgtgctaagaccacccagctgtccctgggttctcatcctagggccttctttgcttccaggtcaggggacctgcttcaatgagaaagcaactgaattgaggctaggagaggtagggagagctgagttctgacttcacctgtgcagaactctctgcccccatgttacctggactggaacagactgtgaatatagcagaaggttccaagaactctggtgtctgacctagaagaggcacagttctctctactggaaagaaaacgatgtagccgattgcacaagggtgccaagggaagacccaggatggcccatcaaaggaacctgggggaggatgcaggaggctgaagggatgcacctggcatttctctcactgtgctcttaccgcatcagcaacccccaacttttgggcctactctgccccccatgcgtgaataccctgcttggatgctgtgcttttccggtttgtctctaagcccctttctccagggcatgttggtttccctggcctctcagtgtcctaactggagcccagagtgccttgttctgagccaggagacggctgagcactggccctccacacctaagcgtcctttacattaacttattggtcttgtataacacctggtgccattgccaagtggctgtgtcctcagctacagagctggaattgtgtggggtttagtgctaaatacttcaataaagtctgttttttgtgattggctg

SEQ ID NO: 6 - DNA, 4828 bp, Homo sapiens
  misc_feature: Gene ID  CDH1
agtggcgtcggaactgcaaagcacctgtgagcttgcggaagtcagttcagactccagcccgctccagcccggcccgacccgaccgcacccggcgcctgccctcgctcggcgtccccggccagccatgggcccttggagccgcagcctctcggcgctgctgctgctgctgcaggtctcctcttggctctgccaggagccggagccctgccaccctggctttgacgccgagagctacacgttcacggtgccccggcgccacctggagagaggccgcgtcctgggcagagtgaattttgaagattgcaccggtcgacaaaggacagcctatttttccctcgacacccgattcaaagtgggcacagatggtgtgattacagtcaaaaggcctctacggtttcataacccacagatccatttcttggtctacgcctgggactccacctacagaaagttttccaccaaagtcacgctgaatacagtggggcaccaccaccgccccccgccccatcaggcctccgtttctggaatccaagcagaattgctcacatttcccaactcctctcctggcctcagaagacagaagagagactgggttattcctcccatcagctgcccagaaaatgaaaaaggcccatttcctaaaaacctggttcagatcaaatccaacaaagacaaagaaggcaaggttttctacagcatcactggccaaggagctgacacaccccctgttggtgtctttattattgaaagagaaacaggatggctgaaggtgacagagcctctggatagagaacgcattgccacatacactctcttctctcacgctgtgtcatccaacgggaatgcagttgaggatccaatggagattttgatcacggtaaccgatcagaatgacaacaagcccgaattcacccaggaggtctttaaggggtctgtcatggaaggtgctcttccaggaacctctgtgatggaggtcacagccacagacgcggacgatgatgtgaacacctacaatgccgccatcgcttacaccatcctcagccaagatcctgagctccctgacaaaaatatgttcaccattaacaggaacacaggagtcatcagtgtggtcaccactgggctggaccgagagagtttccctacgtataccctggtggttcaagctgctgaccttcaaggtgaggggttaagcacaacagcaacagctgtgatcacagtcactgacaccaacgataatcctccgatcttcaatcccaccacgtacaagggtcaggtgcctgagaacgaggctaacgtcgtaatcaccacactgaaagtgactgatgctgatgcccccaataccccagcgtgggaggctgtatacaccatattgaatgatgatggtggacaatttgtcgtcaccacaaatccagtgaacaacgatggcattttgaaaacagcaaagggcttggattttgaggccaagcagcagtacattctacacgtagcagtgacgaatgtggtaccttttgaggtctctctcaccacctccacagccaccgtcaccgtggatgtgctggatgtgaatgaagcccccatctttgtgcctcctgaaaagagagtggaagtgtccgaggactttggcgtgggccaggaaatcacatcctacactgcccaggagccagacacatttatggaacagaaaataacatatcggatttggagagacactgccaactggctggagattaatccggacactggtgccatttccactcgggctgagctggacagggaggattttgagcacgtgaagaacagcacgtacacagccctaatcatagctacagacaatggttctccagttgctactggaacagggacacttctgctgatcctgtctgatgtgaatgacaacgcccccataccagaacctcgaactatattcttctgtgagaggaatccaaagcctcaggtcataaacatcattgatgcagaccttcctcccaatacatctcccttcacagcagaactaacacacggggcgagtgccaactggaccattcagtacaacgacccaacccaagaatctatcattttgaagccaaagatggccttagaggtgggtgactacaaaatcaatctcaagctcatggataaccagaataaagaccaagtgaccaccttagaggtcagcgtgtgtgactgtgaaggggccgccggcgtctgtaggaaggcacagcctgtcgaagcaggattgcaaattcctgccattctggggattcttggaggaattcttgctttgctaattctgattctgctgctcttgctgtttcttcggaggagagcggtggtcaaagagcccttactgcccccagaggatgacacccgggacaacgtttattactatgatgaagaaggaggcggagaagaggaccaggactttgacttgagccagctgcacaggggcctggacgctcggcctgaagtgactcgtaacgacgttgcaccaaccctcatgagtgtcccccggtatcttccccgccctgccaatcccgatgaaattggaaattttattgatgaaaatctgaaagcggctgatactgaccccacagccccgccttatgattctctgctcgtgtttgactatgaaggaagcggttccgaagctgctagtctgagctccctgaactcctcagagtcagacaaagaccaggactatgactacttgaacgaatggggcaatcgcttcaagaagctggctgacatgtacggaggcggcgaggacgactaggggactcgagagaggcgggccccagacccatgtgctgggaaatgcagaaatcacgttgctggtggtttttcagctcccttcccttgagatgagtttctggggaaaaaaaagagactggttagtgatgcagttagtatagctttatactctctccactttatagctctaataagtttgtgttagaaaagtttcgacttatttcttaaagcttttttttttttcccatcactctttacatggtggtgatgtccaaaagatacccaaattttaatattccagaagaacaactttagcatcagaaggttcacccagcaccttgcagattttcttaaggaattttgtctcacttttaaaaagaaggggagaagtcagctactctagttctgttgttttgtgtatataattttttaaaaaaaatttgtgtgcttctgctcattactacactggtgtgtccctctgcctttttttttttttttaagacagggtctcattctatcggccaggctggagtgcagtggtgcaatcacagctcactgcagccttgtcctcccaggctcaagctatccttgcacctcagcctcccaagtagctgggaccacaggcatgcaccactacgcatgactaattttttaaatatttgagacggggtctccctgtgttacccaggctggtctcaaactcctgggctcaagtgatcctcccatcttggcctcccagagtattgggattacagacatgagccactgcacctgcccagctccccaactccctgccattttttaagagacagtttcgctccatcgcccaggcctgggatgcagtgatgtgatcatagctcactgtaacctcaaactctggggctcaagcagttctcccaccagcctcctttttatttttttgtacagatggggtcttgctatgttgcccaagctggtcttaaactcctggcctcaagcaatccttctgccttggccccccaaagtgctgggattgtgggcatgagctgctgtgcccagcctccatgttttaatatcaactctcactcctgaattcagttgctttgcccaagataggagttctctgatgcagaaattattgggctcttttagggtaagaagtttgtgtctttgtctggccacatcttgactaggtattgtctactctgaagacctttaatggcttccctctttcatctcctgagtatgtaacttgcaatgggcagctatccagtgacttgttctgagtaagtgtgttcattaatgtttatttagctctgaagcaagagtgatatactccaggacttagaatagtgcctaaagtgctgcagccaaagacagagcggaactatgaaaagtgggcttggagatggcaggagagcttgtcattgagcctggcaatttagcaaactgatgctgaggatgattgaggtgggtctacctcatctctgaaaattctggaaggaatggaggagtctcaacatgtgtttctgacacaagatccgtggtttgtactcaaagcccagaatccccaagtgcctgcttttgatgatgtctacagaaaatgctggctgagctgaacacatttgcccaattccaggtgtgcacagaaaaccgagaatattcaaaattccaaattttttcttaggagcaagaagaaaatgtggccctaaagggggttagttgaggggtagggggtagtgaggatcttgatttggatctctttttatttaaatgtgaatttcaacttttgacaatcaaagaaaagacttttgttgaaatagctttactgtttctcaagtgttttggagaaaaaaatcaaccctgcaatcactttttggaattgtcttgatttttcggcagttcaagctatatcgaatatagttctgtgtagagaatgtcactgtagttttgagtgtatacatgtgtgggtgctgataattgtgtattttctttgggggtggaaaaggaaaacaattcaagctgagaaaagtattctcaaagatgcatttttataaattttattaaacaattttgttaaaccataaaaaaaaaaaaa

SEQ ID NO: 7 - DNA, 5289 bp, Homo sapiens
  misc_feature: Gene ID  COBL
ccagccccttccgtccgccgcgctccgccgctgtcctccgccggcccgcgtgggaggacgatgggtcagcgcggaaaaagtgaatgagggcggcagcggtggcggcggtagccagcgcctgactcggcgggcccggagcaccgcccgcgtcccggcccccggcaccatggacgcgccgcgcgcctcggcggccaagcccccgaccgggaggaagatgaaggctcgtgctcccccacctcctggaaaggctgccactctgcatgtgcacagtgaccagaagcccccccacgatggggccctcgggtcgcagcagaacttggttcgcatgaaggaggcgctgagggccagcaccatggacgtcaccgtggtcctgcctagcgggctggagaagaggagcgtgctcaatgggagccatgcgatgatggacctactggttgaactttgccttcagaaccacctgaatccatcccaccatgcccttgaaattcggtcttcagaaacccaacaacctttgagttttaagccaaatactttgattgggaccctgaatgtgcatactgtgtttctgaaagaaaaagttcctgaagagaaggttaagcctggtccccctaaggtgcctgagaaatctgtgcgtttggtcgtgaattacctgcggacacaaaaagctgttgtgcgtgtgagccctgaggttcctctccagaatattctcccagtcatttgtgcaaagtgtgaggtcagcccagagcacgtggttctcctcagggacaacattgccggagaggagctggagctgtccaagtccctgaacgagctcgggataaaggagctctacgcgtgggacaacagaagagaaacctttaggaaatcatcacttggcaatgatgagacagataaagagaagaaaaaatttctgggatttttcaaagttaataaaagaagcaatagtaagggctgtttaacgacccccaactccccatccatgcactcacgttctcttacgctgggtccatccctctcgctgggcagcatctcaggggtgtccgtgaagtcggagatgaagaagcgccgagcccctcctcctccaggttcagggccacctgtgcaagacaaggcatcggaaaaggtatctcttgggtcacagattgatttacagaagaagaagcggcgagcgccagctccccctccaccacagccaccaccaccgagtcccctgatccccaaccgcactgaggataaggaggagaacaggaagagcacgatggtaagcctgcccctggggtctggcagccactgcagcccggatggagccccgcaggtgctgtcagaggcggaggagaccgtgtcagttggcagctgttttgcgtcggaggacacgaccgaggactcaggagtgatgagttccccctcagacatcgtctctctggactcgcagcaggacagcatgaaatacaaagacaagtgggccacagaccaggaagactgcagtgaccaggacctcgctggaaccccagacctgggtccccagaagagccccttgtgggagaagaatggctctgagaactcacatctgagaactgaaaaagctgtcacagcctcaaatgatgaagaagacctattaattgctggagagttccgtaaaacccttgcagaacttgatgaagacctggaagaaatggaagacagctatgaaacagacaccagctccctcaccagctccatccatggtgcatccaaccactgcccacaggatgccatgatccctcacggcgacacagatgcaatcccagtaacattcataggggaagtttcagatgatcctgtggattcggggttgttttccaatagaaacaacaatgctgggtctttcgactcggagggtgttgccagcaggagagactccctggcgccacttcaagctgagcacagccagccccatgaaaaggcaagggaggaagtacctgccctgcaccccgcttcccatgacgtcggaaaaggaatccgtgtggccttatctaacatctctaaagatgggaatctaatggaaacggcgcccagggtgacttcttttgcttcgaatcttcacacagacaacctaaatgcaaaagtgaaagacaaagtgtatggctgtgctgacggggagaggactcaagccacagagagagtgaattcccaaccggtgaatgaaaaggacagcaacgataaaaacgctgccttggcaccaacatcatggcaccaacgaggccaaaacccagggaaaagctacagacttaagcacggcctcacaacgtataaaatcattcctccaaagtcagagatgcgatgttacgacagagatgtgtccctctccaccggagccattaagattgacgagctggggaacttggtgagtcctcacgccaccggcatcaggatcatttccctgtcttcgtctgtgcctgaagcagaatctcagcccattgggaaagtcagagagttctggaggtgcaactctgtggaaaaacacctgggccgaccctcagagagctctgccaggggacccccctccacccctgtgcccacgcagacgcagaatccagagagcagactccaagcagaccccaagccaatatcgccccagcagaagtctgcccaccatgagggccggaaccccctaggggaggggagaaaccagccccccaccatgggcatgggtcacgtgagggtgccagcagctcacaccacagaagtcacatttctcaagcctcagagaagaacgtccagccagtatgtggcctctgccattgccaagcgcataggagccccaaaagtccatgctgatgtggtgaggccacacggttatgccgagaagggttatgcaggcaaggcgccagtgctggctgcaccacctgtcactgtgaaagatgacaggacatccagcccacactcagagacacaaggatggaaggatggtgcccagtggccctgtgtcactcctcccaacaaccacggggaagatttggcagtgggagcccctcctaggggggaggtcattggcccacacaggaagttgtctactcaggacagacctgctgctatccacagaagctcctgtttctcactggttcagtcttcccagagggatcgtgtttctgtgggacagagctgtggtttcagtggaaagcaaagcacaagtagccaggaggccagctcagcatctgagcccagacgcgcacctgatggtacagacccaccccctccacacacatctgacactcaggcctgcagcagggagctggtcaatggctctgtgcgcgccccaggccacggcgagccttcccaccctccaggagggtctggcacagaaagccacattctcctagaaagagaggaaaagcccagtgtgttctctacagatggaaatgaaacagacagtatttggccacccagcatttttgggccgaagaaaaaattcaaacctgttgtccagagaccagtcccaaaagacacatccctgcactctgccctgatggaagccatccactctgcgggagggaaggacagactacgcaagacggcagaacacaccggggagggcaggccagcgaaactgtcctacacggaggcagagggcgaacgatctgcactgctggcagctatccgcgggcacagcggcacctgcagcctgaggaaggtggcatcctctgcttctgaggagctccagagcttccgagatgccgcactctctgctcagggctcggaaagtcctctgctagaagaccttggtcttctgtccccaccagccattcccccaccaccccctccaccctcccaggctctctctgcaccaaggacggcctccaggttcagcacgggcaccctcagcaacaccgcagacgcaaggcaagccttgatggacgccatccgctccggcacaggggctgcgagactgagaaaggtgcccttgctcgtgtgaatgaacagaggccagagaattgcagaaacccacctgtaatatgcccacaggcgtcactcaaggagtctgcatagccaggaacatcaagttttggtgttactttcttgtctacaggccaaatgcgcacagttgattttcggtgtgttcctgtataacggcttgaaaggaaaggagaagatgctgtgtcgctgcgcagctcgtgtgccaaggaaatgtgttttgcctctgaatatcttaagttgctaataaacgattcttgttggcaggttaatgggactataaatgctggagcccagcactcacacttaattatatttatgggcaaagtaaaaagaatcatgccttagactgattttggacctttgagacttttaccacatacagatggatgaaactacttactgatttaagatcttttgaattttagaaaaaacatatgaatcagatggaacaatctgaatgtgatatgctccatgatgctgtgtgttttggtagtaagaaaaaaaattgggaggcttttcaaaggtggctttgttaggtagtaaaactgctggattcttcttcctcaaatatttggggctaacacttcacagagggtcacagtccatgatctgaccaactaagcttttaaaaatcaactattaccacccagaaaacagtcctacgtgatcatgtggccagggcatgcccgagtcctgcaagggtggcgggtggctgaggtcacaccaccactggagtggatgggcagtgccccacatggacagccattccccaggctatgaagtgacataggcctcccccacggtgcctgtgtacggagcctgatgacttcactgggagccttctggaatcctgcagagggtcaaggagcagggatgttggatgcccacctgtcaagagttcagatcaaagttgcgctgagagcatcacaattttggttcagccttgacgcgtttgtccaacagctcattggctccctttgtatgatatcgtggtcttctcacatggtgcccagtcaccaatatttataatgaggtctaactacagcagtagtttttcatatatatctctaaaacattttgttatattgaaaaaagtaatagaaatcaagatgtgttgatgaaataaaatgtgtatctgagtgagaaaacaagtatggtgaggtcactttaatgtttcacagcgatctcagatctaggcctcaggtagaatggaagctgttctgcattcactgattaacgttgctaaactcttggtgaggcacgagctaccagccaattgctcttcatcacagctatctgtcttttagtgccacaaataaaagaaaatcaaatctt

SEQ ID NO: 8 - DNA, 2080 bp, Homo sapiens
  misc_feature: Gene ID CYP3A7
tcactgctgtgcagggcaggaaagctccacacacacagcccagcaaacagcagcacgctgctgaaaaaaagactcagaggagagagataaggaaggaaagtagtgatggatctcatcccaaacttggccgtggaaacctggcttctcctggctgtcagcctgatactcctctatctatatggaacccgtacacatggactttttaagaagcttggaattccagggcccacacctctgccttttttgggaaatgctttgtccttccgtaagggctattggacgtttgacatggaatgttataaaaagtatagaaaagtctggggtatttatgactgtcaacagcctatgctggctatcacagatcccgacatgatcaaaacagtgctagtgaaagaatgttattctgtcttcacaaaccggaggcctttcgggccagtgggatttatgaaaaatgccatctctatagctgaggatgaagaatggaagagaatacgatcattgctgtctccaacattcaccagcggaaaactcaaggagatggtccctatcattgcccagtatggagatgtgttggtgagaaatctgaggcgggaagcagagacaggcaagcctgtcaccttgaaacacgtctttggggcctacagcatggatgtgatcactagcacatcatttggagtgagcatcgactctctcaacaatccacaagacccctttgtggaaaacaccaagaagcttttaagatttaatccattagatccattcgttctctcaataaaagtctttccattccttaccccaattcttgaagcattaaatatcactgtgtttccaagaaaagttataagttttctaacaaaatctgtaaaacagataaaagaaggtcgcctcaaagagacacaaaagcaccgagtggatttccttcagctgatgattgactctcagaattcaaaagactctgagacccacaaagctctgtctgatctggagctcatggcccaatcaattatctttatttttgctggctatgaaaccacgagcagtgttctctccttcattatatatgaactggccactcaccctgatgtccagcagaaagtgcagaaggaaattgatacagttttacccaataaggcaccacccacctatgatactgtgctacagttggagtatcttgacatggtggtgaatgaaacactcagattattcccagttgctatgagacttgagagggtctgcaaaaaagatgttgaaatcaatgggatgtttattcccaaaggggtggtggtgatgattccaagctatgttcttcatcatgacccaaagtactggacagagcctgagaagttcctccctgaaaggttcagtaaaaagaacaaggacaacatagatccttacatatacacaccctttggaagtggacccagaaactgcattggcatgaggtttgctctcgtgaacatgaaacttgctctagtcagagtccttcagaacttctccttcaaaccttgtaaagaaacacagatccccctgaaattacgctttggaggacttcttctaacagaaaaacccattgttctaaaggctgagtcaagggatgagaccgtaagtggagcctgatttccctaaggacttctggtttgctctttaagaaagctgtgccccagaacaccagagacctcaaattactttacaaatagaaccctgaaatgaagacgggcttcatccaatgtgctgcataaataatcagggattctgtacgtgcattgtgctctctcatggtctgtatagagtgttatacttggtaatatagaggagatgaccaaatcagtgctggggaagtagatttggcttctctgcttctcataggactatctccaccacccccagttagcaccattaactcctcctgagctctgataacataattaacatttctcaataatttcaaccacaatcattaataaaaataggaattattttgatggctctaacagtgacatttatatcatgtgttatatctgtagtattctatagtaagctttatattaagcaaatcaataaaaacctctttacaaaagta

SEQ ID NO: 9 - DNA, 3516 bp, Homo sapiens
  misc_feature: Gene ID  DSG2
tcgagggtgcgatggcgcggacgcgggaccgcgtacgcctgctgcttctcctgatctgctttaacgttggaagtggacttcacttacaggtcttaagcacaagaaatgaaaataagctgcttcctaaacatcctcatttagtgcggcaaaagcgcgcctggatcaccgcccccgtggctcttcgggagggagaggatctgtccaagaagaatccaattgccaagatacattctgatcttgcagaagaaagaggactcaaaattacttacaaatacactggaaaagggattacagagccaccttttggtatatttgtctttaacaaagatactggagaactgaatgttaccagcattcttgatcgagaagaaacaccattttttctgctaacaggttacgctttggatgcaagaggaaacaatgtagagaaacccttagagctacgcattaaggttcttgatatcaatgacaacgaaccagtgttcacacaggatgtctttgttgggtctgttgaagagttgagtgcagcacatactcttgtgatgaaaatcaatgcaacagatgcagatgagcccaataccctgaattcgaaaatttcctatagaatcgtatctctggagcctgcttatcctccagtgttctacctaaataaagatacaggagagatttatacaaccagtgttaccttggacagagaggaacacagcagctacactttgacagtagaagcaagagatggcaatggagaagttacagacaaacctgtaaaacaagctcaagttcagattcgtattttggatgtcaatgacaatatacctgtagtagaaaataaagtgcttgaagggatggttgaagaaaatcaagtcaatgtagaagttacgcgcataaaagtgttcgatgcagatgaaataggttctgataattggctggcaaattttacatttgcatcaggaaatgaaggaggttatttccacatagaaacagatgctcaaactaacgaaggaattgtgacccttattaaggaagtagattatgaagaaatgaagaatcttgacttcagtgttattgtcgctaataaagcagcttttcacaagtcgattaggagtaaatacaagcctacacccattcccatcaaggtcaaagtgaaaaatgtgaaagaaggcattcattttaaaagcagcgtcatctcaatttatgttagcgagagcatggatagatcaagcaaaggccaaataattggaaattttcaagcttttgatgaggacactggactaccagcccatgcaagatatgtaaaattagaagatagagataattggatctctgtggattctgtcacatctgaaattaaacttgcaaaacttcctgattttgaatctagatatgttcaaaatggcacatacactgtaaagattgtggccatatcagaagattatcctagaaaaaccatcactggcacagtccttatcaatgttgaagacatcaacgacaactgtcccacactgatagagcctgtgcagacaatctgtcacgatgcagagtatgtgaatgttactgcagaggacctggatggacacccaaacagtggccctttcagtttctccgtcattgacaaaccacctggcatggcagaaaaatggaaaatagcacgccaagaaagtaccagtgtgctgctgcaacaaagtgagaaaaagcttgggagaagtgaaattcagttcctgatttcagacaatcagggttttagttgtcctgaaaagcaggtccttacactcacagtttgtgaggttctgcatggcagcggctgcagggaagcacagcatgactcctatgtgggcctgggacccgcagcaattgcgctcatgattttggcctttctgctcctgctattggtaccacttttactgctgatgtgccattgcggaaagggcgccaaagcgtttacccccatacctggcaccatagagatgctgcatccttggaataatgaaggagcaccacctgaagacaaggtggtgccatcatttctgccagtggatcaagggggcagtctagtaggaagaaatggagtaggaggtatggccaaggaagccacgatgaaaggaagtagctctgcttccattgtcaaagggcaacatgagatgtccgagatggatggaaggtgggaagaacacagaagcctgctttctggtagagctacccagtttacaggggccacaggcgctatcatgaccactgaaaccacgaagaccgcaagggccacaggggcttccagagacatggccggagctcaggcagctgctgttgcactgaacgaagaattcttaagaaattatttcactgataaagcggcctcttacactgaggaagatgaaaatcacacagccaaagattgccttctggtttattctcaggaagaaactgaatcgctgaatgcttctattggttgttgcagttttattgaaggagagctagatgaccgcttcttagatgatttgggacttaaattcaagacgctagctgaagtttgcctgggtcaaaaaatagatataaataaggaaattgagcagagacaaaaacctgccacagaaacaagtatgaacacagcttcacattcactctgtgagcaaactatggttaattcagagaatacctactcctctggcagtagcttcccagttccaaaatctttgcaagaagccaatgcagagaaagtaactcaggaaatagtcactgaaagatctgtgtcttctaggcaggcgcaaaaggtagctacacctcttcctgacccaatggcttctagaaatgtgatagcaacagaaacttcctatgtcacagggtccactatgccaccaaccactgtgatcctgggtcctagccagccacagagccttattgtgacagagagggtgtatgctccagcttctaccttggtagatcagccttatgctaatgaaggtacagttgtggtcactgaaagagtaatacagcctcatgggggtggatcgaatcctctggaaggcactcagcatcttcaagatgtaccttacgtcatggtgagggaaagagagagcttccttgcccccagctcaggtgtgcagcctactctggccatgcctaatatagcagtaggacagaatgtgacagtgacagaaagagttctagcacctgcttccactctgcaatccagttaccagattcccactgaaaattctatgacggctaggaacaccacggtgtctggagctggagtccctggccctctgccagattttggtttagaggaatctggtcattctaattctaccataaccacatcttccaccagagtcaccaagcatagcactgtacagcattcttactcctaaacagcagtcagccacaaactgacccagagtttaattagcagtgactaatttcatgtttccaatgtacctgatttttcatgagccttacagacacacagagacacatacacattgatcttaaaatttttctcagtcactgatatgcaaagga

SEQ ID NO: 10 - DNA, 5036 bp, Homo sapiens
  misc_feature: Gene ID  ELF3
gtgctgccgtgtgggcaagctctgtggggagaggggaggccagtgggttgtttttgccatcacagaatactgggaagcccctggcatcctgctccatagctctcttcaccactatcctggaaccttctccccacccccatccccatgcctccaaggcactgacctcaaatccaagtctttcccacttatctcaagctgccagcctgtagggattccttatctcagctccatgtcagcggtgaggaagccccaagaaggcaagggagctgacagccttctcatttttctcgtacatcctcctgttcaccccgccatcccgggagccccagccagatgctcttcagggcagggagcacgtgagcagccctggggctagaagccggttctcccacattcctgggtgagggactgggtggagggtgtgcctgcctcaggctccttgggggaggcccctgaagggctggggaaaatcctactgagccccaggctctcctgcctgcactggcccagtgcgggggcgggggggaggggggatcctggacattaactctctcctaggcacctgggagcttcagagaggaagaaggtaaggtaagaagcagattctctgcgcaggactttcttagtcctcaggctatagagttaaagaaagaagtatctgtagaaggtggagtcaccttttgtttgagtaaccgtttcacccaccctggctgagggtggccaggaactccccaagttgtgcattaatggaggtgggtcacacagatcctacatttttttgtttttgttttttttttttgagacgcagtgattacaggtgtgagccaccggatcctacatttcaaatgcataaaaatctagatatgggctgggcgcagtagctcatgcctgtaatcccagcacattgggaggctgaggcaggcagatcatgaggtcaggagatcgagtccatcctggctaacatggtgaaaccctgtctctactaaaaatacaaaaagccgggcatggcagcgggcgcctgtaatcccagctactcggaaagctgaggcaggagaatcgcttgaacccaggagtcagaggttgcagtgagcagagatcacgccactgcactccaacctgggcgacagagcgagactccacctcaaaacaaaataaacaaaatactagatctggaagagatcttagggattattaaattcagacaacctcattttttatagatggggaaacaagcacagactccaagggcctcatccaagatcacacagttgcagatgctggctacaagtctcctgcctcaaccacctgtattaccccattcagggtctcaagaagggtctataagacactatccattgtgcttcgggctgagtccatagagacaaccacagacatgggggactctgcccacagggaaggcaagggctctggccatggagctggatgggaagactctgaagcccgaagacattgaatcctgtgcagggaaagagcgagggttttgtgtacaacacacctgcatacctggatgtgaatctcagctccaccccttcaccaactctgtgtggcctgggcaagccattctaagggaaccctccacactgcaactttcatgtctataaaatgggaataaccatgcattccttacaggacttttttggtgtgaggattaaatgagagaatatgttgaaaagtgcttggtaaatatattaatactatgcattccctcttctttgaatgacgtgacccaggtagtcaggcttctgaccactagagggcagcagaaggtactggaaaactgggccgagtgaaccagagattagatggggtccagagagcagggatgaacttacccgtgtggattctggcaactccggcagggagggctccagcaggcgctgagggaagaactttcaagcagagccgggtctcttcaggagcgactgcagcaaccctgatgcttggatggagtccaggcaggtgatggtagtgaagaccttgccaacagagtgggcgctggagaaggagccctttagtggggaccctggggccacgactaggctggcaggcccagccagcaccaattaatccatgagtattgcccagcattgagcctggagcaccttccagcccctggccagagtcctgggtgttctgggaaaaacccctaaacctagtaactcctctccctactaggcctctttgttgctgaatctctggaatttaggggccagcagctttctgactcaggtcagccaggggttcatgttccctcacttgccctccccctgcctggcccatctctggcctggcccctgggaggaatttcctgggccagagggcagccgaaagcacagatgcccaccccagcaacgttcccgccacctgcccaggccagtgccccgtgcccaaccccagagggtgcgggatgacagactctgacaatcattaaaccagccgggcctgatttcccagcactgcctgctaagatccgggccaagtggcactgaatatgcaaatcacctggggccaggagcccagtctaaaggccaggaaatcccctccatccaatgagacaccagctcaggttactgcaggggacacactataaagccctgagctcagggaggagctccctccaggctctatttagagccgggtaggggagcgcagcggccagatacctcagcgctacctggcggaactggatttctctcccgcctgccggcctgcctgccacagccggactccgccactccggtaggattccccgcctgtcattccctagcccagctcttgggaaactgcagaggggtccagaggatttgcagttctgaacctgcacactccagtctaggatctccgagcaagagcgtaggtgtcctgagggtcaaagaacagagagagattgtctctgggaaggcagaatggccatgacgccgctagcctggctccagggccccagagatctgaggagggaagcccagctggaggctcctgtggtcctgccctggtctgagatcttggagcccttcttgaagagagggtgtccgcagagttgctgatcttcctgcccctgggggctactcttgcccagggttgggcaaagcagagtagctgggagtgtaaggagaggaccctcgtcccctcaccaacctcatcctctctccccctacccacgggtagcctcatggctgcaacctgtgagattagcaacatttttagcaactacttcagtgcgatgtacagctcggaggactccaccctggcctctgttccccctgctgccacctttggggccgatgacttggtactgaccctgagcaacccccagatgtcattggagggtacagagaaggccagctggttgggggaacagccccagttctggtcgaagacgcaggttctggactggatcagctaccaagtggagaagaacaagtacgacgcaagcgccattgacttctcacgatgtgacatggatggcgccaccctctgcaattgtgcccttgaggagctgcgtctggtctttgggcctctgggggaccaactccatgcccagctgcgagacctcacttccagctcttctgatgagctcagttggatcattgagctgctggagaaggatggcatggccttccaggaggccctagacccagggccctttgaccagggcagcccctttgcccaggagctgctggacgacggtcagcaagccagcccctaccaccccggcagctgtggcgcaggagccccctcccccggcagctctgacgtctccaccgcagggactggtgcttctcggagctcccactcctcagactccggtggaagtgacgtggacctggatcccactgatggcaagctcttccccagcgatggttttcgtgactgcaagaagggggatcccaagcacggggagcggaaacgaggccggccccgaaagctgagcaaagagtactgggactgtctcgagggcaagaagagcaagcacgcgcccagaggcacccacctgtgggagttcatccgggacatcctcatccacccggagctcaacgagggcctcatgaagtgggagaatcggcatgaaggcgtcttcaagttcctgcgctccgaggctgtggcccaactatggggccaaaagaaaaagaacagcaacatgacctacgagaagctgagccgggccatgaggtactactacaaacgggagatcctggaacgggtggatggccggcgactcgtctacaagtttggcaaaaactcaagcggctggaaggaggaagaggttctccagagtcggaactgagggttggaactatacccgggaccaaactcacggaccactcgaggcctgcaaaccttcctgggaggacgggcaggccagatggcccctccactggggaatgctcccagctgtgctgtggagagaagctgatgttttggtgtattgtcagccatcgtcctgggactcggagactatggcctcgcctccccaccctcctcttggaattacaagccctggggtttgaagctgactttatagctgcaagtgtatctccttttatctggtgcctcctcaaacccagtctcagacactaaatgcagacaacaccttcctcctgcagacacctggactgagccaaggaggcctggggaggccctaggggagcaccgtgatggagaggacagagcaggggctccagcaccttctttctggactggcgttcacctccctgctcagtgcttgggctccacgggcaggggtcagagcactccctaatttatgtgctatataaatacgtcagatgtacatagagatctattttttctaaaacattcccctccccactcctctcccacagagtgctggactgttccaggccctccagtgggctgatgctgggacccttaggatggggctcccagctcctttctcctgtgaatggaggcagagacctccaataaagtgccttctgggctttttctaaaaaaaaaaaaaaaaaaaaaaaaaaagaaaaaaaaaaaaaaaaaaaaca

SEQ ID NO: 11 - DNA, 5511 bp, Homo sapiens; identical to SEQ ID NO: 4
  misc_feature: Gene ID  ERBB3

SEQ ID NO: 12 - DNA, 1654 bp, Homo sapiens
  misc_feature: Gene ID  KCTD14
ggctgcgcagtggagcggccagtgggcaggatgacgagccagacccctctgccccagtccccccggcccaggcggccgacgatgtctactgttgtggagctgaacgtcgggggtgagttccacaccaccaccctgggtaccctgaggaagtttccgggctcaaagctggcagagatgttctctagcttagccaaggcctccacggacgcggagggccgcttcttcatcgaccgccccagcacctatttcagacccatcctggactacctgcgcactgggcaagtgcccacacagcacatccctgaagtgtaccgtgaggctcagttctacgaaatcaagcctttggtcaagctgctggaggacatgccacagatctttggtgagcaggtgtctcggaagcagtttttgctgcaagtgccgggctacagcgagaacctggagctcatggtgcgcctggcacgtgcagaagccataacagcacggaagtccagcgtgcttgtgtgcctggtggaaactgaggagcaggatgcatattattcagaggtcctgtgttttctgcaggataagaagatgttcaagtctgttgtcaagtttgggccctggaaggcggtcctagacaacagcgacctcatgcactgcctggagatggacattaaggcccaggggtacaaggtattctccaagttctacctgacgtaccccaccaaaagaaacgaattccattttaacatttattcattcaccttcacctggtggtgatcctcaggagcagagactgttatgaattctggcgtggcttatgaaattaaaagttgccatcaaagccattttcttttaatttcacaaacatcaggcaatttccagggttggtctagagtcttgccactaaatattgatcactcgtttaaggactttccactccattgcaactgatgccactatatttgcctagcaacttgcagctacttccttttcaaagcctcatgtatctcccagacccttctcttgaagtccaataacaagaccaagtaagaatgtttcaacaatgcgttggcaagagatgtgagatgacaacaggaacatacaagatactgtgaatctagatgttctgacccaaagatgtagtctacatagccccagcttggggtccaatccatctgtccctggcatgtgccttcatgtagtaggtgctttcctgatcccctttgcgagatgctgtgggtgctaacacctcagagctgtcctcttctctagagtgaaggttttcagagtgcatcatcagcattacctgtgaacttgctggaaatacaaatcctcaggccccacctcagacctactgaatcagaatctctgggggttggcacagcattctgatttaccaaaccctccaagtgattttgatgtattctaattttgagaccatctctagaaaagaattgctacctcttgtatggaggtacaaaagactgacctcttacatcaaggaacttcctttcccagagctcctcatggaatcaagctgaagtcagtcttcttctgagagcacattcttactcagtttttttcctctgtcctacgctgcttccctcactccccttctcctaagagcactccatcaataaaccacttgcacgagaaaaaaaaaaaaaaaaaa

SEQ ID NO: 13 - DNA, 3350 bp, Homo sapiens
  misc_feature: Gene ID KLF5
tagtcgcggggcaggtacgtgcgctcgcggttctctcgcggaggtcggcggtggcgggagcgggctccggagagcctgagagcacggtggggcggggcgggagaaagtggccgcccggaggacgttggcgtttacgtgtggaagagcggaagagttttgcttttcgtgcgcgccttcgaaaactgcctgccgctgtctgaggagtccacccgaaacctcccctcctccgccggcagccccgcgctgagctcgccgacccaagccagcgtgggcgaggtgggaagtgcgcccgacccgcgcctggagctgcgcccccgagtgcccatggctacaagggtgctgagcatgagcgcccgcctgggacccgtgccccagccgccggcgccgcaggacgagccggtgttcgcgcagctcaagccggtgctgggcgccgcgaatccggcccgcgacgcggcgctcttccccggcgaggagctgaagcacgcgcaccaccgcccgcaggcgcagcccgcgcccgcgcaggccccgcagccggcccagccgcccgccaccggcccgcggctgcctccagaggacctggtccagacaagatgtgaaatggagaagtatctgacacctcagcttcctccagttcctataattccagagcataaaaagtatagacgagacagtgcctcagtcgtagaccagttcttcactgacactgaagggttaccttacagtatcaacatgaacgtcttcctccctgacatcactcacctgagaactggcctctacaaatcccagagaccgtgcgtaacacacatcaagacagaacctgttgccattttcagccaccagagtgaaacgactgcccctcctccggccccgacccaggccctccctgagttcaccagtatattcagctcacaccagaccgcagctccagaggtgaacaatattttcatcaaacaagaacttcctacaccagatcttcatctttctgtccctacccagcagggccacctgtaccagctactgaatacaccggatctagatatgcccagttctacaaatcagacagcagcaatggacactcttaatgtttctatgtcagctgccatggcaggccttaacacacacacctctgctgttccgcagactgcagtgaaacaattccagggcatgcccccttgcacatacacaatgccaagtcagtttcttccacaacaggccacttactttcccccgtcaccaccaagctcagagcctggaagtccagatagacaagcagagatgctccagaatttaaccccacctccatcctatgctgctacaattgcttctaaactggcaattcacaatccaaatttacccaccaccctgccagttaactcacaaaacatccaacctgtcagatacaatagaaggagtaaccccgatttggagaaacgacgcatccactactgcgattaccctggttgcacaaaagtttataccaagtcttctcatttaaaagctcacctgaggactcacactggtgaaaagccatacaagtgtacctgggaaggctgcgactggaggttcgcgcgatcggatgagctgacccgccactaccggaagcacacaggcgccaagcccttccagtgcggggtgtgcaaccgcagcttctcgcgctctgaccacctggccctgcatatgaagaggcaccagaactgagcactgcccgtgtgacccgttccaggtcccctgggctccctcaaatgacagacctaactattcctgtgtaaaaacaacaaaaacaaacaaaagcaagaaaaccacaactaaaactggaaatgtatattttgtatatttgagaaaacagggaatacattgtattaataccaaagtgtttggtcattttaagaatctggaatgcttgctgtaatgtatatggctttactcaagcagatctcatctcatgacaggcagccacgtctcaacatgggtaaggggtgggggtggaggggagtgtgtgcagcgtttttacctaggcaccatcatttaatgtgacagtgttcagtaaacaaatcagttggcaggcaccagaagaagaatggattgtatgtcaagattttacttggcattgagtagtttttttcaatagtaggtaattccttagagatacagtatacctggcaattcacaaatagccattgaacaaatgtgtgggtttttaaaaattatatacatatatgagttgcctatatttgctattcaaaattttgtaaatatgcaaatcagctttataggtttattacaagttttttaggattcttttggggaagagtcataattcttttgaaaataaccatgaatacacttacagttaggatttgtggtaaggtacctctcaacattaccaaaatcatttctttagagggaaggaataatcattcaaatgaactttaaaaaagcaaatttcatgcactgattaaaataggattattttaaatacaaaaggcattttatatgaattataaactgaagagcttaaagatagttacaaaatacaaaagttcaacctcttacaataagctaaacgcaatgtcatttttaaaaagaaggacttagggtgtcgttttcacatatgacaatgttgcatttatgatgcagtttcaagtaccaaaacgttgaattgatgatgcagttttcatatatcgagatgttcgctcgtgcagtactgttggttaaatgacaatttatgtggattttgcatgtaatacacagtgagacacagtaattttatctaaattacagtgcagtttagttaatctattaatactgactcagtgtctgcctttaaatataaatgatatgttgaaaacttaaggaagcaaatgctacatatatgcaatataaaatagtaatgtgatgctgatgctgttaaccaaagggcagaataaataagcaaaatgccaaaaggggtcttaattgaaatgaaaatttaattttgtttttaaaatattgtttatctttatttattttgtggtaatatagtaagtttttttagaagacaattttcataacttgataaattatagttttgtttgttagaaaagttgctcttaaaagatgtaaatagatgacaaacgatgtaaataattttgtaagaggcttcaaaatgtttatacgtggaaacacacctacatgaaaagcagaaatcggttgctgttttgcttctttttccctcttatttttgtattgtggtcatttcctatgcaaataatggagcaaacagctgtatagttgtagaattttttgagagaatgagatgtttatatattaacgacaattttttttttggaaaataaaaagtgcctaaaaga

SEQ ID NO: 14 - DNA, 1788 bp, Homo sapiens
  misc_feature: Gene ID  KRT8
attcctgagagctctcctcaccaagaagcagcttctccgctccttctaggatctccgcctggttcggcccgcctgcctccactcctgcctctaccatgtccatcagggtgacccagaagtcctacaaggtgtccacctctggcccccgggccttcagcagccgctcctacacgagtgggcccggttcccgcatcagctcctcgagcttctcccgagtgggcagcagcaactttcgcggtggcctgggcggcggctatggtggggccagcggcatgggaggcatcaccgcagttacggtcaaccagagcctgctgagcccccttgtcctggaggtggaccccaacatccaggccgtgcgcacccaggagaaggagcagatcaagaccctcaacaacaagtttgcctccttcatagacaaggtacggttcctggagcagcagaacaagatgctggagaccaagtggagcctcctgcagcagcagaagacggctcgaagcaacatggacaacatgttcgagagctacatcaacaaccttaggcggcagctggagactctgggccaggagaagctgaagctggaggcggagcttggcaacatgcaggggctggtggaggacttcaagaacaagtatgaggatgagatcaataagcgtacagagatggagaacgaatttgtcctcatcaagaaggatgtggatgaagcttacatgaacaaggtagagctggagtctcgcctggaagggctgaccgacgagatcaacttcctcaggcagctatatgaagaggagatccgggagctgcagtcccagatctcggacacatctgtggtgctgtccatggacaacagccgctccctggacatggacagcatcattgctgaggtcaaggcacagtacgaggatattgccaaccgcagccgggctgaggctgagagcatgtaccagatcaagtatgaggagctgcagagcctggctgggaagcacggggatgacctgcggcgcacaaagactgagatctctgagatgaaccggaacatcagccggctccaggctgagattgagggcctcaaaggccagagggcttccctggaggccgccattgcagatgccgagcagcgtggagagctggccattaaggatgccaacgccaagttgtccgagctggaggccgccctgcagcgggccaagcaggacatggcgcggcagctgcgtgagtaccaggagctgatgaacgtcaagctggccctggacatcgagatcgccacctacaggaagctgctggagggcgaggagagccggctggagtctgggatgcagaacatgagtattcatacgaagaccaccagcggctatgcaggtggtctgagctcggcctatgggggcctcacaagccccggcctcagctacagcctgggctccagctttggctctggcgcgggctccagctccttcagccgcaccagctcctccagggccgtggttgtgaagaagatcgagacacgtgatgggaagctggtgtctgagtcctctgacgtcctgcccaagtgaacagctgcggcagcccctcccagcctacccctcctgcgctgccccagagcctgggaaggaggccgctatgcagggtagcactgggaacaggagacccacctgaggctcagccctagccctcagcccacctggggagtttactacctggggaccccccttgcccatgcctccagctacaaaacaattcaattgctttttttttttggtccaaaataaaacctcagctagctctgccaatgtc

SEQ ID NO: 15 - DNA, 4018 bp, Homo sapiens
  misc_feature: Gene ID MAP7
gttccgcccgccgctcttccagctgagactcggcagcggcggtcggagcggctgccacgccgcacctgcccgcgagggggcgcgccaggcccaggccgcagccgactgccgcgctctctgtccccgccctccgccatccccggactgcgctcacctgtctgggccgctggcctgggaggcgggggccggcgggagccaagccgaggaaagggcggagcggctctccgggcgcgtcatcggagcaccatggcggagctaggagctggcggcgacggccacaggggcggcgacggcgcagtgcgaagcgaaacagcacccgacagctacaaagtgcaagataagaaaaatgcctccagccgccctgcctctgcaatttcaggacaaaataacaaccactcaggaaataaaccagaccctccgcctgtgttacgtgttgatgaccggcagcggctggcccgggagcgacgtgaggaacgggagaaacagctagctgcaagagaaatagtgtggttagaaagagaagagcgagccaggcagcactacgagaagcacctggaagagcggaagaagaggttggaggagcagaggcagaaggaggagcggaggagggctgctgtggaggagaagcggaggcagagacttgaggaggacaaagaacgccacgaagctgttgtacggcgcacaatggaaaggagccagaagccaaaacagaagcataaccgttggtcgtggggaggctctctccatgggagccctagcatccacagtgcagatccagacaggcggtcagtttccaccatgaatctttcgaaatatgttgatcccgtcattagcaagcggctctcctcttcatctgcaactttactaaattctccagatagagctcgccgcctgcagctcagcccatgggagagcagcgttgttaacagactcctgacgcccacacattcgttcctggccagaagtaaaagcacagctgccttgtctggagaagcagcatcttgcagccccatcatcatgccctacaaagctgcacactctagaaattcgatggatcgaccaaaactctttgtaacaccacctgagggctcttctcgcaggaggatcattcatggcacagcgagctataaaaaagaaagagagagagaaaatgtactcttcctcacatctggcacccgaagggctgtatctccatctaatcccaaagcaagacaaccagctcgctcccgactttggcttccgtccaagtctcttcctcatttgcctggcacacccagaccgacatcctccttgccacccggctcagtcaaagctgctcctgctcaggtccggcccccatcccccggcaacatccgccctgtcaagagggaagtcaaagtggagcctgagaagaaagatcctgagaaggaacctcagaaagttgccaatgagccctcactaaagggcagagcacctttagtgaaggtagaagaagccacagttgaagagcggacacctgctgaaccagaagttggccctgctgctccagccatggccccagctccagcctcggccccagctccagcctcggccccagctccagccccggtccccaccccagccatggtctcagccccgtcatccactgtgaatgccagtgcttctgttaagacttctgcaggcaccaccgacccagaggaggccacaaggcttctagctgagaagaggcggctggcccgagagcagagagaaaaggaagaaagggagaggagggagcaggaagagcttgaaagacaaaagagagaggaattggctcaacgtgtggctgaagagaggacgactcgccgtgaggaggagtcgcgcaggctggaagccgagcaggcccgggagaaggaggagcagctgcagcggcaggcggaggagcgggcgctgcgcgagcgggaggaggcagagcgcgcccagaggcagaaagaagaagaagctcgcgttcgtgaagaagcagagagggtccggcaggaacgagagaagcatttccagagagaagagcaagagcgcctggagagaaagaagcgacttgaggagattatgaaaagaaccaggagaacagaagctacagataagaaaaccagtgatcagagaaacggtgatatagccaagggagctctcactggaggaacagaggtgtctgcacttccatgtacaacaaacgctccgggaaatggaaagccagttggcagcccacatgtggttacctcacaccagtcaaaagtgacagtggagagcactcccgatttggaaaaacaaccaaatgaaaatggtgtatctgttcagaatgaaaattttgaagaaattataaacttacccattggatctaaaccatccagattagatgtcaccaacagtgagagcccagaaattcctttgaatccaattttggcctttgatgatgaagggacacttgggcccctgcctcaggtagatggtgttcagacacagcagactgcagaagttatatgagtgtttcttctgaagaaccaaagctgaaatttaatgagaatttctacaattaatggaattcctttcctgctataaaggagcatcccctccacccgttttctagagttcttgaccatcattttgaaaagatttattaaaactagctaaagacaacagactggatagcttttctaataattttcatcaataggaaaaaagaaatacgtctcattcttcaatactttaaaatggctttttccagtgtgctccttcttagcaatcaatatttttctgcattctttaaaagacaagagaatttggttataaaagaaatgggctgactaggcatgatttttttggtcttaaaagcttaacatgtaaaattggcaaaaaaaattttttaccttttataatacttgaaaaataagtacctctttgttctacaagtagaatgaataggagaagagtttaagcctgtttttttaaaatattattgcaaagagctctatttgtagaagcaaattataggcagattaccaggttcttataaatacagcttgtacatggacattctgcaaacccagctgtcacatttttcttgcaactccttttgcaaaagcagactaaaatgttttaaaatgtgaaaaaacattattttttcaaagcaagaaaataatttactgccctcttacataatgtatttataaagtttttccagataaactaatcaaataaattagaataatgtgacaacattacaaatttaatttgttagctgcattccttctgatgttaccacgatagaatgttactgatgattcagggctatttctgaagtctgtatgttgctgctgtccccagtgatggtggacttatctttgccttacctgatcacaaattatgttggggaaaataaagatttaatatttctttaaatagaaaaagaatttggttttgctcgtttaagagcaatgagaaaatgatggaatgttgactgtgtttggcacacaggacacggaccttcatggaagtccttgctctgcgtggcatctgtcagcttttcacctttcattcttattcttcacttttgctgctgagcctagctgtacaaacttgcactttcatttgctaatataaattcagttttattttaccattttagagactactaatgattaaatgtagaaggagagggtgcacatgtttttatgtggagtgtttaaaagataaatttataccactgtaatgtgcagcttttattaaaagagaaattggttgaactgctaggttgaatgagagacttcatctattggactattttttttaatccaggcatatggtctttagtaatggcttgtaatttgtgaaaacattaatttgggggttttccctgttttcagttgtccatgtacacatagtcattatattagaaaagaaagctgttcaacaaacttgtttaatttgtttaaatcaacatagcatgaaacaccaaataaaatgtttgacatagttttaaaaaaaaaaaaaaaaaaaaaaaaaaaaa

SEQ ID NO: 16 - DNA, 2601 bp, Homo sapiens
  misc_feature: Gene ID NQO1
ccgcccttgtaggctgtccacctcaaacgggccggacaggatatataagagagaatgcaccgtgcactacacacgcgactcccacaaggttgcagccggagccgcccagctcaccgagagcctagttccggccagggtcgccccggcaaccacgagcccagccaatcagcgccccggactgcaccagagccatggtcggcagaagagcactgatcgtactggctcactcagagaggacgtccttcaactatgccatgaaggaggctgctgcagcggctttgaagaagaaaggatgggaggtggtggagtcggacctctatgccatgaacttcaatcccatcatttccagaaaggacatcacaggtaaactgaaggaccctgcgaactttcagtatcctgccgagtctgttctggcttataaagaaggccatctgagcccagatattgtggctgaacaaaagaagctggaagccgcagaccttgtgatattccagttccccctgcagtggtttggagtccctgccattctgaaaggctggtttgagcgagtgttcataggagagtttgcttacacttacgctgccatgtatgacaaaggacccttccggagtaagaaggcagtgctttccatcaccactggtggcagtggctccatgtactctctgcaagggatccacggggacatgaatgtcattctctggccaattcagagtggcattctgcatttctgtggcttccaagtcttagaacctcaactgacatatagcattgggcacactccagcagacgcccgaattcaaatcctggaaggatggaagaaacgcctggagaatatttgggatgagacaccactgtattttgctccaagcagcctctttgacctaaacttccaggcaggattcttaatgaaaaaagaggtacaggatgaggagaaaaacaagaaatttggcctttctgtgggccatcacttgggcaagtccatcccaactgacaaccagatcaaagctagaaaatgagattccttagcctggatttccttctaacatgttatcaaatctgggtatctttccaggcttccctgacttgctttagtttttaagatttgtgtttttctttttccacaaggaataaatgagagggaatcgactgtattcgtgcatttttggatcatttttaactgattcttatgattactatcatggcatataaccaaaatccgactgggctcaagaggccacttagggaaagatgtagaaagatgctagaaaaatgttctttaaaggcatctacacaatttaattcctctttttagggctaaagttttagggtacagtttggctaggtatcattcaactctccaatgttctattaatcacctctctgtagtttatggcagaagggaattgctcagagaaggaaaagactgaatctacctgccctaagggacttaacttgtttggtagttagccatctaatgcttgtttatgatatttcttgctttcaattacaaagcagttactaatatgcctagcacaagtaccactcttggtcagcttttgttgtttatatacagtacacagataccttgaaaggaagagctaataaatctcttctttgctgcagtcatctactttttttttaattaaaaaaaatttttttttgaagcagtcttgctctgttacccaggctggagtgcagtggtgtgatctcggctcactgcaacctctgcctcccaggttccagcaattctcctgcctcagcctccctagtagctgggatgacaggcgcctgccatcatgcctgactaatttttgtatttttagtagagacggcgtttcaccatgttggccaggctggtctcaaactcctgacctcaggtgatccgcctacctcagcctcccaaagtgctgggattacaggcgtgatccaccacacctggcccttgcaatcttctactttaaggtttgcagagataaaccaataaatccacaccgtacatctgcaatatgaattcaagaaaggaaatagtaccttcaatacttaaaaatagtcttccacaaaaaatactttatttctgatctatacaaattttcagaaggttattttctttatcattgctaaactgatgacttactatgggatggggtccagtcccatgaccttggggtacaattgtaaacctagagttttatcaactttggtgaacagttttggcataatagtcaatttctacttctggaagtcatctcattccactgttggtattatataattcaaggagaatatgataaaacactgccctcttgtggtgcattgaaagaagagatgagaaatgatgaaaaggttgcctgaaaaatgggagacagcctcttacttgccaagaaaatgaagggattggaccgagctggaaaacctcctttaccagatgctgactggcactggtggtttttgctctcgacagtatccacaatagctgacggctgggtgtttcagtttgaaaatattttgttgccttcatcttcactgcaattttgtgtaaatttctcaaagatctgaattaaataaataaaattcatttctacagacccacaaaaaaaaaaa

SEQ ID NO: 17 - DNA, 2499 bp, Homo sapiens
  misc_feature: Gene ID NQO1
ccgcccttgtaggctgtccacctcaaacgggccggacaggatatataagagagaatgcaccgtgcactacacacgcgactcccacaaggttgcagccggagccgcccagctcaccgagagcctagttccggccagggtcgccccggcaaccacgagcccagccaatcagcgccccggactgcaccagagccatggtcggcagaagagcactgatcgtactggctcactcagagaggacgtccttcaactatgccatgaaggaggctgctgcagcggctttgaagaagaaaggatgggaggtggtggagtcggacctctatgccatgaacttcaatcccatcatttccagaaaggacatcacaggtaaactgaaggaccctgcgaactttcagtatcctgccgagtctgttctggcttataaagaaggccatctgagcccagatattgtggctgaacaaaagaagctggaagccgcagaccttgtgatattccagttccccctgcagtggtttggagtccctgccattctgaaaggctggtttgagcgagtgttcataggagagtttgcttacacttacgctgccatgtatgacaaaggacccttccggagtggcattctgcatttctgtggcttccaagtcttagaacctcaactgacatatagcattgggcacactccagcagacgcccgaattcaaatcctggaaggatggaagaaacgcctggagaatatttgggatgagacaccactgtattttgctccaagcagcctctttgacctaaacttccaggcaggattcttaatgaaaaaagaggtacaggatgaggagaaaaacaagaaatttggcctttctgtgggccatcacttgggcaagtccatcccaactgacaaccagatcaaagctagaaaatgagattccttagcctggatttccttctaacatgttatcaaatctgggtatctttccaggcttccctgacttgctttagtttttaagatttgtgtttttctttttccacaaggaataaatgagagggaatcgactgtattcgtgcatttttggatcatttttaactgattcttatgattactatcatggcatataaccaaaatccgactgggctcaagaggccacttagggaaagatgtagaaagatgctagaaaaatgttctttaaaggcatctacacaatttaattcctctttttagggctaaagttttagggtacagtttggctaggtatcattcaactctccaatgttctattaatcacctctctgtagtttatggcagaagggaattgctcagagaaggaaaagactgaatctacctgccctaagggacttaacttgtttggtagttagccatctaatgcttgtttatgatatttcttgctttcaattacaaagcagttactaatatgcctagcacaagtaccactcttggtcagcttttgttgtttatatacagtacacagataccttgaaaggaagagctaataaatctcttctttgctgcagtcatctactttttttttaattaaaaaaaatttttttttgaagcagtcttgctctgttacccaggctggagtgcagtggtgtgatctcggctcactgcaacctctgcctcccaggttccagcaattctcctgcctcagcctccctagtagctgggatgacaggcgcctgccatcatgcctgactaatttttgtatttttagtagagacggcgtttcaccatgttggccaggctggtctcaaactcctgacctcaggtgatccgcctacctcagcctcccaaagtgctgggattacaggcgtgatccaccacacctggcccttgcaatcttctactttaaggtttgcagagataaaccaataaatccacaccgtacatctgcaatatgaattcaagaaaggaaatagtaccttcaatacttaaaaatagtcttccacaaaaaatactttatttctgatctatacaaattttcagaaggttattttctttatcattgctaaactgatgacttactatgggatggggtccagtcccatgaccttggggtacaattgtaaacctagagttttatcaactttggtgaacagttttggcataatagtcaatttctacttctggaagtcatctcattccactgttggtattatataattcaaggagaatatgataaaacactgccctcttgtggtgcattgaaagaagagatgagaaatgatgaaaaggttgcctgaaaaatgggagacagcctcttacttgccaagaaaatgaagggattggaccgagctggaaaacctcctttaccagatgctgactggcactggtggtttttgctctcgacagtatccacaatagctgacggctgggtgtttcagtttgaaaatattttgttgccttcatcttcactgcaattttgtgtaaatttctcaaagatctgaattaaataaataaaattcatttctacagacccacaaaaaaaaaaa

SEQ ID NO: 18 - DNA, 2487 bp, Homo sapiens
  misc_feature: Gene ID NQO1
ccgcccttgtaggctgtccacctcaaacgggccggacaggatatataagagagaatgcaccgtgcactacacacgcgactcccacaaggttgcagccggagccgcccagctcaccgagagcctagttccggccagggtcgccccggcaaccacgagcccagccaatcagcgccccggactgcaccagagccatggtcggcagaagagcactgatcgtactggctcactcagagaggacgtccttcaactatgccatgaaggaggctgctgcagcggctttgaagaagaaaggatgggaggtggtggagtcggacctctatgccatgaacttcaatcccatcatttccagaaaggacatcacaggtaaactgaaggaccctgcgaactttcagtatcctgccgagtctgttctggcttataaagaaggccatctgagcccagatattgtggctgaacaaaagaagctggaagccgcagaccttgtgatattccagagtaagaaggcagtgctttccatcaccactggtggcagtggctccatgtactctctgcaagggatccacggggacatgaatgtcattctctggccaattcagagtggcattctgcatttctgtggcttccaagtcttagaacctcaactgacatatagcattgggcacactccagcagacgcccgaattcaaatcctggaaggatggaagaaacgcctggagaatatttgggatgagacaccactgtattttgctccaagcagcctctttgacctaaacttccaggcaggattcttaatgaaaaaagaggtacaggatgaggagaaaaacaagaaatttggcctttctgtgggccatcacttgggcaagtccatcccaactgacaaccagatcaaagctagaaaatgagattccttagcctggatttccttctaacatgttatcaaatctgggtatctttccaggcttccctgacttgctttagtttttaagatttgtgtttttctttttccacaaggaataaatgagagggaatcgactgtattcgtgcatttttggatcatttttaactgattcttatgattactatcatggcatataaccaaaatccgactgggctcaagaggccacttagggaaagatgtagaaagatgctagaaaaatgttctttaaaggcatctacacaatttaattcctctttttagggctaaagttttagggtacagtttggctaggtatcattcaactctccaatgttctattaatcacctctctgtagtttatggcagaagggaattgctcagagaaggaaaagactgaatctacctgccctaagggacttaacttgtttggtagttagccatctaatgcttgtttatgatatttcttgctttcaattacaaagcagttactaatatgcctagcacaagtaccactcttggtcagcttttgttgtttatatacagtacacagataccttgaaaggaagagctaataaatctcttctttgctgcagtcatctactttttttttaattaaaaaaaatttttttttgaagcagtcttgctctgttacccaggctggagtgcagtggtgtgatctcggctcactgcaacctctgcctcccaggttccagcaattctcctgcctcagcctccctagtagctgggatgacaggcgcctgccatcatgcctgactaatttttgtatttttagtagagacggcgtttcaccatgttggccaggctggtctcaaactcctgacctcaggtgatccgcctacctcagcctcccaaagtgctgggattacaggcgtgatccaccacacctggcccttgcaatcttctactttaaggtttgcagagataaaccaataaatccacaccgtacatctgcaatatgaattcaagaaaggaaatagtaccttcaatacttaaaaatagtcttccacaaaaaatactttatttctgatctatacaaattttcagaaggttattttctttatcattgctaaactgatgacttactatgggatggggtccagtcccatgaccttggggtacaattgtaaacctagagttttatcaactttggtgaacagttttggcataatagtcaatttctacttctggaagtcatctcattccactgttggtattatataattcaaggagaatatgataaaacactgccctcttgtggtgcattgaaagaagagatgagaaatgatgaaaaggttgcctgaaaaatgggagacagcctcttacttgccaagaaaatgaagggattggaccgagctggaaaacctcctttaccagatgctgactggcactggtggtttttgctctcgacagtatccacaatagctgacggctgggtgtttcagtttgaaaatattttgttgccttcatcttcactgcaattttgtgtaaatttctcaaagatctgaattaaataaataaaattcatttctacagacccacaaaaaaaaaaa

SEQ ID NO: 19 - DNA, 4021 bp, Homo sapiens
  misc_feature: Gene ID  RBM35B
ggtagccgccccgccccgcggggcgccacgggcgggtcttggcagcgcccactgagccagccgggccgcaggtgccgcccccgatacacggtgtcccgcccaagctgatccgcgtctgcggtcggtcggtgcgtgcgtgcgcctcgtcggtccgcgtgtctggccgagagcccccttcctctgcggccatgactccgccgccgccgccgccccctcccccgggccctgaccccgcggccgaccccgccgcggacccctgcccctggcccggatcactggtcgtcctcttcggggctacggcgggtgcgctgggacgggacctgggctcggacgagaccgacttaatcctcctagtttggcaagtggttgagccgcggagccgccaggtggggacgctgcacaaatcgctggttcgtgccgaggcggccgcactgagtacgcagtgccgcgaggcgagcggcctgagcgccgacagcctggcgcgggcagagccgctggacaaggtgctgcagcagttctcacagctggtgaacggggatgtggctttgctgggcgggggcccctacatgctctgcactgatgggcagcagctattgcgacaggtcctgcaccccgaggcctccaggaagaacctggtgctccccgacatgttcttctccttctatgacctccgaagagaattccatatgcagcatccaagcacctgccctgccagggacctcactgtggccaccatggcacagggtttaggactggagacagatgccacagaggatgactttggggtctgggaagtcaagacaatggtagctgttatcctccatctactcaaagagcccagcagtcaattgttttcgaagcccgaggtgataaagcagaaatacgagacggggccttgcagcaaggctgatgtggtggacagtgagactgtggtacgggctcgtgggttgccgtggcagtcatcagaccaggacgtggctcgcttcttcaaagggctcaacgtggccaggggtggtgtagcactctgcctcaacgcccagggccgcagaaatggcgaggccctcatccgctttgtggacagcgagcagcgggacctagcgctgcagagacacaagcaccacatgggcgtccgctatattgaggtgtataaagcgacaggggaggagtttgtaaagattgcagggggcacatcactagaggtggctcgtttcttgtcacgggaagaccaagtgatcctgcggctgcggggactgcccttctcggctgggccaacggacgtgcttggcttcctggggccagagtgcccagtgactgggggtaccgaggggctgctctttgtgcgccatcctgatggccggccgactggtgatgccttcgccctctttgcttgtgaggagctggcacaggctgcactgcgcaggcacaagggcatgctgggtaagcgatacattgaactcttccggagcactgcagccgaagtgcagcaggtcttgaaccgctatgcatccggcccactccttcctacactgactgccccactgctgcccatccccttcccactggcacctgggactgggagggactgtgtacgcctccgaggcctgccctacacggccaccattgaagacatcctgagctttctgggggaggcagcagctgacattcggccccacggtgtacacatggtgctcaaccagcagggccggccatcgggcgatgccttcattcagatgacatcagcagagcgagccctagctgctgctcagcgttgccataagaaggtgatgaaggagcgctacgtggaggtggtcccctgttccacagaggagatgagccgagtgctgatggggggcaccttgggccgcagtggcatgtcccctccaccctgcaagctgccctgcctctcaccacctacctacaccaccttccaagccaccccaacgctcattcccacggagacggcagctctatacccctcttcagcactgctcccagctgccagggtgcctgctgcccccacccctgttgcctactatccagggccagccactcaactctacctgaactacacagcctactacccaagccccccagtctcccccaccactgtgggctacctcactacacccactgctgccctggcctctgctcccacctcagtgttgtcccagtcaggagccttggtccgcatgcagggtgtcccatacacggctggtatgaaggatctgctcagcgtcttccaggcctaccagctacccgctgatgactacaccagtctgatgcctgttggtgacccacctcgcactgtgttacaagcccccaaggaatgggtgtgtttgtaggagagaaagccaggaggtaagagccagctgatatcctcggcgaacatgtctctcctgagtccagaagaccagcaccctcaacctggtagcttctttctggcttgtcaaagctctcagaaggtacctagaggagcccaagccccagctccatcctccacttattctgcctgtttcccccaaagacaatggctggaccctgcatgcagggctgggggtggaatggggctaaccagctcctgatggcctgagccaggcatcttgactggcacctggagagcccttaagtctgtcctggctgtggcccatgccgacagatatcgtggggctgacaggtccacggcaggcttgctttcttttataaaatggaagctctggtaccttcaatgtatgactcctgggagaatcaagggtccatctgagcctctgagtaaagatcccaatgttctacctctccctgtccctcttgtaggggatagggaggcagagagagccagcccctaccctcagagtatctggacctcagagaccatgttgtgccaggggtggtcccacctaaagatgctagcccctctccaggtgggcataaggagtaacagatggcaaaaccacaaactattttgatggactgtgctgcagtatcaccagaagacattagggggcagtaggcccccacacaaaaccttcaggcttgaattttaaaggggaggactttctgccaacttttcttgtatgccttgggaaagccagttgccctgaacccagcagacaccatggaatgtcctttgcacgcattaaatggtacagaactgaagcctcggaagcaatttggaactcgatcttctcttccttaaatgaaaagttattgaccaaatggactttttaaaagacacaggacccttaactttgccccaaagtgaggggctccacaccaaccccaggcggaggaacactcagacagattaaggatactgttgacctgtcactgtttattatttcagcactaaaactgaggagcctcaactgctggctcttcttccctttgtatttgtgtaaggagcactgcactcccataaaaggttttaaaatacaaaatgtacaagaacacacaattccaagtgctgtaaacataactgagaaccagttcctttactaaacatccattttataaaacacaaggtttcaatttgagcccatctgagccttaaagatccattctgaataccaaaaacagggcttcacagccaggcccagaagaggtctggtgataatggctggccctgggtggggatagtttacacccgggcagcagcaccacacatgaacccaaagacatgttctttttaaagctgttttcagccatgtttctctgtgcatctccagtaagcagaaggctacccattccattcctcaacccaagagctagcacagttagagtaggagggggtgcgtactagcacgtgcccagttgctcagtgctgctagtagaaattgatttgcatagtccaatggatgtgtgctttaacaccactatgttgcacaaaaatttaagtctttatctacaaagccaaaaaatattgactcttaacaccaaagcttttacaaagctgatataaaactgcttacatagtatacaaagctctattttaaaatttaatgtttattttaaataggaaagcatt

SEQ ID NO: 20 - DNA, 3226 bp, Homo sapiens
  misc_feature: Gene ID TMPRSS
cgcgagctaagcaggaggcggaggcggaggcggagggcgaggggcggggagcgccgcctggagcgcggcaggtcatattgaacattccagatacctatcattactcgatgctgttgataacagcaagatggctttgaactcagggtcaccaccagctattggaccttactatgaaaaccatggataccaaccggaaaacccctatcccgcacagcccactgtggtccccactgtctacgaggtgcatccggctcagtactacccgtcccccgtgccccagtacgccccgagggtcctgacgcaggcttccaaccccgtcgtctgcacgcagcccaaatccccatccgggacagtgtgcacctcaaagactaagaaagcactgtgcatcaccttgaccctggggaccttcctcgtgggagctgcgctggccgctggcctactctggaagttcatgggcagcaagtgctccaactctgggatagagtgcgactcctcaggtacctgcatcaacccctctaactggtgtgatggcgtgtcacactgccccggcggggaggacgagaatcggtgtgttcgcctctacggaccaaacttcatccttcagatgtactcatctcagaggaagtcctggcaccctgtgtgccaagacgactggaacgagaactacgggcgggcggcctgcagggacatgggctataagaataatttttactctagccaaggaatagtggatgacagcggatccaccagctttatgaaactgaacacaagtgccggcaatgtcgatatctataaaaaactgtaccacagtgatgcctgttcttcaaaagcagtggtttctttacgctgtatagcctgcggggtcaacttgaactcaagccgccagagcaggatcgtgggcggtgagagcgcgctcccgggggcctggccctggcaggtcagcctgcacgtccagaacgtccacgtgtgcggaggctccatcatcacccccgagtggatcgtgacagccgcccactgcgtggaaaaacctcttaacaatccatggcattggacggcatttgcggggattttgagacaatctttcatgttctatggagccggataccaagtagaaaaagtgatttctcatccaaattatgactccaagaccaagaacaatgacattgcgctgatgaagctgcagaagcctctgactttcaacgacctagtgaaaccagtgtgtctgcccaacccaggcatgatgctgcagccagaacagctctgctggatttccgggtggggggccaccgaggagaaagggaagacctcagaagtgctgaacgctgccaaggtgcttctcattgagacacagagatgcaacagcagatatgtctatgacaacctgatcacaccagccatgatctgtgccggcttcctgcaggggaacgtcgattcttgccagggtgacagtggagggcctctggtcacttcgaagaacaatatctggtggctgataggggatacaagctggggttctggctgtgccaaagcttacagaccaggagtgtacgggaatgtgatggtattcacggactggatttatcgacaaatgagggcagacggctaatccacatggtcttcgtccttgacgtcgttttacaagaaaacaatggggctggttttgcttccccgtgcatgatttactcttagagatgattcagaggtcacttcatttttattaaacagtgaacttgtctggctttggcactctctgccattctgtgcaggctgcagtggctcccctgcccagcctgctctccctaaccccttgtccgcaaggggtgatggccggctggttgtgggcactggcggtcaagtgtggaggagaggggtggaggctgccccattgagatcttcctgctgagtcctttccaggggccaattttggatgagcatggagctgtcacctctcagctgctggatgacttgagatgaaaaaggagagacatggaaagggagacagccaggtggcacctgcagcggctgccctctggggccacttggtagtgtccccagcctacctctccacaaggggattttgctgatgggttcttagagccttagcagccctggatggtggccagaaataaagggaccagcccttcatgggtggtgacgtggtagtcacttgtaaggggaacagaaacatttttgttcttatggggtgagaatatagacagtgcccttggtgcgagggaagcaattgaaaaggaacttgccctgagcactcctggtgcaggtctccacctgcacattgggtggggctcctgggagggagactcagccttcctcctcatcctccctgaccctgctcctagcaccctggagagtgcacatgccccttggtcctggcagggcgccaagtctggcaccatgttggcctcttcaggcctgctagtcactggaaattgaggtccatgggggaaatcaaggatgctcagtttaaggtacactgtttccatgttatgtttctacacattgctacctcagtgctcctggaaacttagcttttgatgtctccaagtagtccaccttcatttaactctttgaaactgtatcacctttgccaagtaagagtggtggcctatttcagctgctttgacaaaatgactggctcctgacttaacgttctataaatgaatgtgctgaagcaaagtgcccatggtggcggcgaagaagagaaagatgtgttttgttttggactctctgtggtcccttccaatgctgtgggtttccaaccaggggaagggtcccttttgcattgccaagtgccataaccatgagcactactctaccatggttctgcctcctggccaagcaggctggtttgcaagaatgaaatgaatgattctacagctaggacttaaccttgaaatggaaagtcttgcaatcccatttgcaggatccgtctgtgcacatgcctctgtagagagcagcattcccagggaccttggaaacagttggcactgtaaggtgcttgctccccaagacacatcctaaaaggtgttgtaatggtgaaaacgtcttccttctttattgccccttcttatttatgtgaacaactgtttgtctttttttgtatcttttttaaactgtaaagttcaattgtgaaaatgaatatcatgcaaataaattatgcgatttttttttcaaagcaaaaaaaaaaaaaaaaaaaaaaaaaaaaaaa

SEQ ID NO: 21 - DNA, 25 bp, Homo sapiens
  misc_feature: Gene ID ANXA3 ; Taken from Table 4 of the Specification
tcagaggaattggaactgatgagaa

SEQ ID NO: 22 - DNA, 25 bp, Homo sapiens
  misc_feature: Gene ID AP1M2; Taken from Table 4 of the Specification
agggcaagccattgatcagccgcaa

SEQ ID NO: 23 - DNA, 25 bp, Homo sapiens
  misc_feature: Gene ID ATP1B1; Taken from Table 4 of the Specification
cagtgcactggcaagcgagatgaag

SEQ ID NO: 24 - DNA, 25 bp, Homo sapiens
  misc_feature: Gene ID C11orf9; Sequence taken from Table 4 of the Specification
gtcatggccttcagcgtggtgtcca

SEQ ID NO: 25 - DNA, 25 bp, Homo sapiens
  misc_feature: Gene ID C1orf106; Taken from Table 4 of the Specification
tgggacagctggggaagctccaggc

SEQ ID NO: 26 - DNA, 25 bp, Homo sapiens
  misc_feature: Gene ID CDH1; Taken from Table 4 of the Specification
cccgccccatcaggcctccgtttct

SEQ ID NO: 27 - DNA, 25 bp, Homo sapiens
  misc_feature: Gene ID COBL; Taken from Table 4 of the Specification
ctgagaaaggtgcccttgctcgtgt

SEQ ID NO: 28 - DNA, 25 bp, Homo sapiens
  misc_feature: Gene ID CYP3A7; Taken from Table 4 of the Specification
ttattttgatggctctaacagtgac

SEQ ID NO: 29 - DNA, 25 bp, Homo sapiens
  misc_feature: Gene ID DSG2; Taken from Table 4 of the Specification
cagagaggaacacagcagctacact

SEQ ID NO: 30 - DNA, 25 bp, Homo sapiens
  misc_feature: Gene ID ELF3; Taken from Table 4 of the Specification
caagcacgcgcccagaggcacccac

SEQ ID NO: 31 - DNA, 25 bp, Homo sapiens
  misc_feature: Gene ID ERBB3; Taken from Table 4 of the Specification
gcaactctcaggcagtgtgtcctgg

SEQ ID NO: 32 - DNA, 25 bp, Homo sapiens
  misc_feature: Gene ID KLF5; Taken from Table 4 of the Specification
actgcgattaccctggttgcacaaa

SEQ ID NO: 33 - DNA, 25 bp, Homo sapiens
  misc_feature: Gene ID MAP7; Taken from Table 4 of the Specification
gcgaagcgaaacagcacccgacagc

SEQ ID NO: 34 - DNA, 25 bp, Homo sapiens
  misc_feature: Gene ID NQO1; Taken from Table 4 of the Specification
gagccatggtcggcagaagagcact

SEQ ID NO: 35 - DNA, 25 bp, Homo sapiens
  misc_feature: Gene ID RBM35B; Taken from Table 4 of the Specification
gtgctgcagcagttctcacagctgg

SEQ ID NO: 36 - DNA, 25 bp, Homo sapiens
  misc_feature: Gene ID TMPRSS2; Taken from Table 4 of the Specification
tcggtgtgttcgcctctacggacca

(19) United States
(12) Patent Application Publication
(10) Pub. No.: US 2007/0207467 A1
(43) Pub. Date: Sep. 6, 2007

(54) Detection of lymph node metastasis from gastric carcinoma

(76) Inventors: Ming Xu, Berkeley Heights, NJ (US); Jadwiga Markiewicz, Kearny, NJ (US); Renata T. Zieba, Garfield, NJ (US); George Green, Newton, NJ (US); Sean Wuxiong Cao, Three Bridges, NJ (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/365,630

(22) Filed: Mar. 1, 2006

(51) Int. Cl. C12Q 1/68; G01N 33/574

(52) U.S. Cl. 435/6; 435/7.23

(57) ABSTRACT

Methods, compositions and articles are directed to diagnosing the lymph node metastasis from gastric carcinoma, differentiating between negative lymph nodes and lymph nodes with metastasis from gastric carcinoma, testing tissue samples of for determining gastric carcinoma staging status.

### BACKGROUND OF THE INVENTION

There are about 876,000 new stomach cancer cases each year worldwide (Shibuya, Mathers et al. 2002). In the United States, the estimated new cases and deaths in 2005 for stomach cancer are 21,860 and 11,550 respectively (Jemal, Murray et al. 2005). Gastric carcinoma is the most common type (90% to 95%) of stomach cancer, which is followed in the order of incidence by lymphomas, carcinoids and mesenchymal spindle cell tumors (Kumar, Cotran et al. 2003).

Complete local regional control is the key treatment for surviving gastric cancer, which includes total or subtotal gastrectomy, sentinel lymph node dissection, extended lymph node dissection and resection of adjacent tissues affected by metastasis such as splenectomy and pancreatectomy (Hartgrink and van de Velde 2005; Kitagawa, Fujii et al. 2005). Out of all the patients who suffer relapse after curative surgery, up to 87.5% of them are caused by local recurrence or remained regional lymph node metastasis (Songun, Bonenkamp et al. 1996).

In a study covering 148 patients with gastro-esophageal junction (GEJ) carcinomas, it was concluded that the presence of micro metastasis in local lymph nodes is a key indicator of a poor prognosis (Heeren, Kelder et al. 2005). The current prevalent staging/diagnosis of lymph node metastasis from gastric cancer is achieved by palpation and intra-operative frozen section examination. Frozen section examination of lymph nodes can exhibit sensitivity of 74%(Kitagawa, Fujii et al. 2005).

With the appropriate gene markers, gene expression analysis and detection technology such as RT-PCR or quantitative PCR could provide an alternative method for the diagnosis or prognosis of gastric cancer. These methods have potential to provide better sensitivity and accuracy for metastasis detection test. The previously reported gene markers include CK19 (Matsuda, Kitagawa et al. 2004), Carcinoembryonic antigen (CEA), cytokeratin-20 (CK-20), and MAGE-3 (Okada, Fujiwara et al. 2001), CK18, MUC1, hTRT (Ajisaka and Miwa 2003). Unfortunately, as published data shows, most of them are not consistently expressed in all of the tumor tissues and tumor cell lines tested. Markers could not be detected by RT-PCR in 15% (CEA) to over 70% (MAGE-3) of the lymph nodes with metastasis from gastric cancer confirmed by histological examination (Okada, Fujiwara et al. 2001). CK20 was used as a marker for RT-PCR tests for disseminated cancer cells in blood from pancreas, colon, stomach, and lung carcinoma (Chausovsky, Luchansky et al. 1999; Chen, Chen et al. 2004).

Here, markers are identified that are consistently expressed in lymph node with metastasis from gastric carcinoma at easily detectable levels which can be differentiated from their background expression in normal lymph nodes. These markers are useful for the detection of disseminated carcinoma cells from gastric carcinoma, or carcinoma of similar cell origin, such as carcinoma of the esophagus, pancreas, and intestine.

### SUMMARY OF THE INVENTION

In one aspect of the invention, a set of 18 gene markers are identified for detection of lymph node metastasis from gastric carcinoma. These marker genes are expressed at high levels in normal stomach tissues, and the expressions are maintained well in gastric carcinoma with different differentiation status while the background expression levels of these genes are relatively low in normal lymph nodes. In lymph nodes with metastasis from gastric carcinoma, the expression levels of these gene markers are distinctively higher than that of normal lymph nodes.

In another aspect of the invention, methods of diagnosing lymph node metastasis from gastric carcinoma involve obtaining biological samples from a patient and measuring the expression levels of genes selected from a group of Markers, where the gene expression levels above pre-determined cut-off levels are indicative of metastasis from gastric carcinoma. Preferably, the biological samples are lymph nodes samples.

In another aspect of the invention, Markers can be used individually or in combination in assays to detect lymph node metastasis from gastric carcinoma, which may be used to help clinical decision-making.

In another aspect of the invention, Mene markers can be used individually or in combination in assays to identify the origin of metastasis of carcinoma that has a similar origin as gastric carcinoma, such as carcinoma of the pancreas, esophagus, or intestine.

In another aspect of the invention, Markers could be used individually or in combination to collect, detect and identify disseminated cancer cells in blood from gastric carcinoma, or carcinoma with similar tissue origins, such as cancer of the pancreas, esophagus, or intestine.

In another aspect of the invention, Markers are used individually or in combination in immunocytochemistry study for detection of metastasis from gastric carcinoma, or carcinoma with similar tissue origins, such as cancer of the pancreas, esophagus, or intestine.

In yet another aspect of the invention, Markers can be assayed intraoperatively. The intraoperative assays can be conducted by a method that includes the steps of: preparing RNA from a lymph node that drains from the stomach; performing a quantitative RT-PCR method specific to one or more Markers and determining if the presence of the Marker exceeds a predetermined cut-off. The cut-off value can be an absolute value or a value relative to the expression of a control gene(s).

The present invention encompasses microarray or gene chips for performing the methods provided herein.

### BRIEF DESCRIPTION OF THE DRAWINGS

FIG. 1 is a representation of the hierarchical clustering analysis of microarray results (HG U133A, signal from all probe sets) from 10 gastric carcinoma, 10 normal stomach tissue and 6 normal lymph nodes (shown as LN) (7 chips for normal lymph nodes, one sample was run twice on chips) from Example 1, Marker Identification.

FIG. 2.is a representation of the hierarchical clustering analysis of microarray results (HG U133A, signal from 20 selected marker gene probe sets) from 10 gastric carcinoma, 10 normal stomach tissue and 6 normal lymph nodes (7 chips for normal lymph nodes, one sample was run twice on chips). The plot shows the chip signal levels of 20 probe sets (representing 18 genes) among samples of gastric carcinoma, normal stomach tissue and normal lymph node. The distinctive difference in gene signal levels between the normal lymph node (shown as LN) group and the gastric carcinoma/normal stomach tissues is illustrated from Example 1, Marker Identification.

FIG. 3A and 3B show the average and standard error of the identified marker gene signals for Confirmation Study (1), which covers the microarray study of 6 normal lymph nodes and 3 lymph nodes with confirmed metastasis from gastric carcinoma described in Example 2, Confirmation Study (1)—microarray test.

FIG. 4 is a graphical portrayal of raw Ct results of a PCR study that covers a total of 10 normal lymph nodes from 6 different patients, and 16 lymph nodes with histologically confirmed metastasis from gastric carcinoma from 13 different patients. The average raw Ct and standard error for each marker genes are shown. The data was obtained as described in Example 2, Confirmation Study (2)—Realtime RT-PCR Test.

FIG. 5 is a graphical portrayal of normalized Ct results of a PCR study that covers a total of 10 normal lymph nodes from 6 different patients, and 16 lymph nodes with histologically confirmed metastasis from gastric carcinoma from 13 different patients. Ct values were normalized based on the house keeping gene HPRT1 Ct results. The average normalized Ct and standard error for each marker genes are shown. The data was obtained according to Example 2, Confirmation Study (2)—Realtime RT-PCR Test.

### DETAILED DESCRIPTION

The inventive methods, compositions, articles, and kits described and claimed in this specification include one or more Markers. “Marker” is used throughout this specification to refer to:
a) Genes and gene expression products such as mRNA and corresponding cDNA, peptides, proteins, fragments and complements of each of the foregoing, and
b) Compositions such as probes, antibodies, ligands, haptens, and labels that, through physical or chemical interaction with a) indicate the expression of the gene or presence of the gene expression product and wherein the gene, gene expression product or compositions correspond with:
   i) SEQ ID NO ______,
   ii) A combination of SEQ ID NO ______ and a member of the group consisting of SEQ ID NO ______, SEQ ID NO ______, SEQ ID NO ______, SEQ ID NO ______,
   iii) A combination of SEQ ID NO ______ and all of SEQ ID NO ______, SEQ ID NO ______, SEQ ID NO ______, SEQ ID NO ______,
   iv) SEQ ID NO ______-SEQ ID NO ______, or
   v) SEQ ID NO ______-SEQ ID NO ______.

A gene corresponds to the sequence designated by a SEQ ID NO when it contains that sequence. A gene segment or fragment corresponds to the sequence of such gene when it contains a portion of the referenced sequence or its complement sufficient to distinguish it as being the sequence of the gene. A gene expression product corresponds to such sequence when its RNA, mRNA, or cDNA hybridizes to the composition having such sequence (e.g. a probe) or, in the case of a peptide or protein; it is encoded by such mRNA. A segment or fragment of a gene expression product corresponds to the sequence of such gene or gene expression product when it contains a portion of the referenced gene expression product or its complement sufficient to distinguish it as being the sequence of the gene or gene expression product.

Markers for ii and iii are preferred. Markers for iv and v are most preferred.

Sample Preparation.

The distribution of metastases and micrometastases in tissues is not uniform in nodes or other tissues. Therefore, a sufficiently large sample should be obtained so that metastases will not be missed. One approach to the sampling issue in the present method is to homogenize a large tissue sample, and subsequently an appropriate portion of the homogenate is to be used to prepare RNA for molecular testing.

Preferably, the samples used in the methods of the invention are lymph node samples from lymph nodes that drain from the stomach. A useful procedure for preparing such a sample for RT-PCR assay follows; an Omni homogenizer and disposable probe for homogenization are used followed by purification of RNA with RNeasy (Qiagen) kit reagents:

Homogenization
a) Determine the sample weight in milligrams, if not previously recorded. Place a fresh piece of wax paper on a balance, tare, and weigh the sample. Preferably, nodes samples are greater than 30 mg.
b) Using a fresh scalpel, mince all tissue from the same node into pieces approximately 4 mm in diameter. Care is taken to avoid contamination of the tissue during processing.
c) Add homogenization (RLT) buffer to the polypropylene homogenization tube.

Preferably, use 2 mL homogenization buffer per 100 mg tissue.
d) Using a clean forceps, transfer the tissue into the homogenization buffer.
e) Place a new homogenization probe onto the manual homogenizer.
f) Homogenize each node completely.
g) Process the homogenate per the RNA Purification procedures in standard Qiagen protocol, RNA is stored at −65° C. or below before use.
h) Dispose of the homogenization probe.
i) Store any remaining homogenate at −65° C. or below for possible later use.
   Diagnostic Assays

The Markers of this invention are useful for providing important clinical information such as whether the gastric carcinoma has metastasized to lymph node, which is indicative to the clinical staging of gastric carcinoma and the prognosis for the patient. Collectively, this information is referred to as “Diagnostic Information” in this specification. Additionally, such Diagnostic Information includes its use as a component in providing therapy. For example, a physician having such Diagnostic Information can base his therapy (e.g., adjuvant therapy) on an indication that the cancer has metastasized to lymph nodes.

Preferred methods for measuring the Markers of the invention include determining the amount of mRNA that is produced by a gene that can code for a protein or peptide. This can be accomplished by competitive reverse transcriptase PCR (RT-PCR), real time RT-PCR, Northern Blot analysis or other tests with similar capabilities. When a large number of Markers of the invention are to be measured simultaneously, it is preferable to generate amplified complementary RNA (cRNA) from mRNA (RNA) and then apply it to DNA microarray for testing and analysis. A number of different array configurations and methods for their production are known to those of skill in the art and are described in U.S. Patents such as: U.S. Pat. Nos. 5,445,934; 5,532,128; 5,556,752; 5,242,974; 5,384,261; 5,405,783; 5,412,087; 5,424,186; 5,429,807; 5,436,327; 5,472,672; 5,527,681; 5,529,756; 5,545,531; 5,554,501; 5,561,071; 5,571,639; 5,593,839; 5,599,695; 5,624,711; 5,658,734; and 5,700,637.

Microarray technology allows for the measurement of the steady-state mRNA level of thousands of genes simultaneously thereby presenting a powerful tool for identifying effects such as the onset, arrest, or modulation of uncontrolled cell proliferation. Although differences exist in the construction of microarray chips, the downstream data analysis and output are similar. The product of these analyses are typically measurements of the intensity of the signal received from a probe set used to detect a target sequence prepared from the sample that hybridizes to a nucleic acid sequence at a known location on the microarray. Typically, the intensity of the signal is proportional to the quantity of target prepared, and thus mRNA, expressed in the sample cells. A large number of such techniques are available and useful. Preferred methods for determining gene expression can be found in U.S. Pat. Nos. 6,271,002; 6,218,122; 6,218,114; and 6,004,755.

In one embodiment, portfolios of genes are established such that a combination of genes in the portfolio exhibit improved sensitivity and specificity relative to individual genes or randomly selected combinations of genes. In the context of the instant invention, the sensitivity of the portfolio is preferably reflected in the fold differences exhibited by a gene's expression in the diseased state relative to the normal state. Specificity is preferably reflected in statistical measurements of the correlation of the signaling of gene expression with the parameter one is interested in determining (cancerous, metastatic, likely to recur, etc.). For example, standard deviation can be a used as such a measurement. In considering a group of genes for inclusion in a portfolio, a small standard deviation in expression measurements correlates with greater specificity. Other measurements of variation such as correlation coefficients can also be used in this capacity. The invention also encompasses the above methods where the specificity is at least about 70%, preferably at least about 80% and more preferably at least about 90%. The invention also encompasses the above methods where the sensitivity is at least about 80% and preferably at least 90%.

Analysis of the expression levels of genes can be conducted by comparing signal intensities of microarrays. This is best done by generating a ratio matrix of the expression intensities of genes in a test sample versus those in a control sample. For instance, the gene expression intensities from a diseased tissue can be compared with the expression intensities generated from benign or normal tissue of the same type. A ratio of these expression intensities indicates the fold-change in gene expression between the test and control samples.

The invention also encompasses the above methods where the comparison of expression patterns is conducted with pattern recognition methods. One method of the invention involves comparing gene expression profiles for various genes (or portfolios) to ascribe diagnoses. The gene expression profiles of each of the genes comprising the portfolio are fixed in a medium such as a computer readable medium. This can take a number of forms. For example, a table can be established into which the range of signals (e.g., intensity measurements) indicative of disease is input. Actual patient data can then be compared to the values in the table to determine whether the patient samples are normal, benign or diseased. In a more sophisticated embodiment, patterns of the expression signals (e.g., fluorescent intensity) are recorded digitally or graphically. The gene expression patterns from the gene portfolios used in conjunction with patient samples are then compared to the expression patterns.

Pattern comparison software can then be used to determine whether the patient samples have a pattern indicative of the disease. Of course, these comparisons can also be used to determine whether the patient is not likely to experience the disease. The expression profiles of the samples are then compared to the portfolio of a control cell. If the sample expression patterns are consistent with the expression pattern for cancer then (in the absence of countervailing medical considerations) the patient is treated as one would treat a gastric cancer patient. If the sample expression patterns are consistent with the expression pattern from the normal/control cell then the patient is diagnosed negative for cancer.

Numerous well-known methods of pattern recognition are available to compare patterns consistent with metastasis to those produced by patient samples. The following references provide some examples: Weighted Voting: Golub et al. (1999); Support Vector Machines: Su et al. (2001); and Ramaswamy et al. (2001); K-nearest Neighbors: Ramaswamy (2001); and Correlation Coefficients: van 't Veer et al. (2002).

Preferably, levels of up and down regulation are distinguished based on fold changes of the intensity measurements of hybridized microarray probes. A 1.5 fold difference is preferred for making such distinctions (or a p-value less than 0.05). That is, before a gene is said to be differentially expressed in diseased versus normal cells, the diseased cell is found to yield at least about 1.5 times more, or 1.5 times less intensity than the normal cells. The greater the fold difference, the more preferred is use of the gene as a diagnostic or prognostic tool. Genes selected for the gene expression profiles of this invention have expression levels that result in the generation of a signal that is distinguishable from those of the normal or non-modulated genes by an amount that exceeds background using clinical laboratory instrumentation.

Statistical values can be used to confidently distinguish modulated from non-modulated genes and noise. Statistical tests find the genes most significantly different between diverse groups of samples. The Student's T-test is an example of a robust statistical test that can be used to find significant differences between two groups. The lower the p-value, the more compelling the evidence that the gene is showing a difference between the different groups. Nevertheless, since microarrays measure more than one gene at a time, tens of thousands of statistical tests may be asked at one time. Because of this, one is unlikely to see small p-values just by chance and adjustments for this using a Sidak correction as well as a randomization/permutation experiment can be made. A p-value less than 0.05 by the T-test is evidence that the gene is significantly different. More compelling evidence is a p-value less then 0.05 after the Sidak correction is factored in. For a large number of samples in each group, a p-value less than 0.05 after the randomization/permutation test is the most compelling evidence of a significant difference.

The present invention encompasses microarrays or gene chips for performing the methods provided herein. The microarrays can contain isolated nucleic acid sequences, their complements, or portions thereof corresponding to Markers where the combination is sufficient to characterize gastric cancer or risk of relapse in a biological sample. The microarray preferably measures or characterizes at least about 1.5-fold over- or under-expression, provides a statistically significant p-value over- or under-expression, or a p-value is less than 0.05. Preferably, the microarray contains a cDNA array or an oligonucleotide array and may contain one or more internal control reagents.

Gene expression profiles can also be displayed in a number of ways. One of the most common method is to arrange raw fluorescence intensities or ratio matrix into a graphical dendogram where columns indicate test samples and rows indicate genes. The data are arranged so genes that have similar expression profiles are proximal to each other. The expression ratio for each gene is visualized as a color. For example, a ratio less than one (indicating down-regulation) may appear in the blue portion of the spectrum while a ratio greater than one (indicating up-regulation) may appear as a color in the red portion of the spectrum. Commercially available computer software programs are available to display such data including “GENESPRING” from Silicon Genetics, Inc. and “DISCOVERY” and “INFER” software from Partek, Inc.

Modulated genes of the invention are described in the Examples. The genes that are differentially expressed are up regulated in lymph nodes with metastasis from gastric carcinoma relative to those lymph nodes without metastasis conditions. Up regulation is a relative term meaning that a detectable difference (beyond the contribution of noise in the system used to measure it) is found in the amount of expression of the genes relative to some baseline. In this case, the baseline is the measured gene expression of makers in normal lymph nodes without metastasis from gastric carcinoma. The genes of interest in the tested lymph node samples are then either up regulated or unchanged relative to the baseline level using the same measurement method. A diagnosis or prognosis includes the determination of disease/status issues such as determining the likelihood of relapse and type of therapy. Determining the likelihood of relapse involves comparing the patterns with patterns associated with known relapse and/or known non-relapsing events or exceeding a cutoff value (usually a Ct in the case of PCR) set for such a distinction.

Genes can be grouped so that information obtained about the set of genes in the group provides a sound basis for making a clinically relevant judgment such as a diagnosis, prognosis, or treatment choice. These sets of genes make up the portfolios of the invention. As with most diagnostic markers, it is often desirable to use the fewest number of markers sufficient to make a correct medical judgment. This prevents a delay in treatment pending further analysis as well unproductive use of time and resources. One method of establishing gene expression portfolios is through the use of optimization algorithms such as the mean variance algorithm widely used in establishing stock portfolios. This method is described in detail in US patent publication number 20030194734. Essentially, the method calls for the establishment of a set of inputs (stocks in financial applications, expression as measured by intensity here) that will optimize the return (e.g., signal that is generated) one receives for using it while minimizing the variability of the return. Many commercial software programs are available to conduct such operations. “Wagner Associates Mean-Variance Optimization Application,” referred to as “Wagner Software” throughout this specification, is preferred. This software uses functions from the “Wagner Associates Mean-Variance Optimization Library” to determine an efficient frontier and optimal portfolios in the Markowitz sense is preferred. Use of this type of software requires that microarray data be transformed so that it can be treated as an input in the way stock return and risk measurements are used when the software is used for its intended financial analysis purposes.

The process of selecting a portfolio can also include the application of heuristic rules. Preferably, such rules are formulated based on biology and an understanding of the technology used to produce clinical results. More preferably, they are applied to output from the optimization method. For example, the mean variance method of portfolio selection can be applied to microarray data for a number of genes differentially expressed in subjects with cancer. Output from the method would be an optimized set of genes that could include some genes that are expressed in peripheral blood as well as in diseased tissue. If samples used in the testing method are obtained from peripheral blood and certain genes differentially expressed in instances of cancer could also be differentially expressed in peripheral blood, then a heuristic rule can be applied in which a portfolio is selected from the efficient frontier excluding those that are differentially expressed in peripheral blood. Of course, the rule can be applied prior to the formation of the efficient frontier by, for example, applying the rule during data pre-selection.

Other heuristic rules can be applied that are not necessarily related to the biology in question. For example, one can apply a rule that only a prescribed percentage of the portfolio can be represented by a particular gene or group of genes. Commercially available software such as the Wagner Software readily accommodates these types of heuristics. This can be, useful, for example, when factors other than accuracy and precision (e.g., anticipated licensing fees) have an impact on the desirability of including one or more genes.

The gene expression profiles of this invention can also be used in conjunction with other non-genetic diagnostic methods useful in cancer diagnosis, prognosis, or treatment monitoring. For example, in some circumstances it is beneficial to combine the diagnostic power of the gene expression based methods described above with data from conventional markers such as serum protein markers (e.g., Cancer Antigen 27.29 (“CA 27.29”)). In one such method, blood is periodically taken from a treated patient and then subjected to an enzyme immunoassay for one of the serum markers described above.

When the concentration of the marker suggests the return of tumors or failure of therapy, a sample source amenable to gene expression analysis is taken. Alternatively, tissue samples may be taken from areas adjacent to the tissue from which a tumor was previously removed. This approach can be particularly useful when other testing produces ambiguous results.

The present invention encompasses methods of generating a gastric cancer diagnostic patient report and reports obtained thereby, by obtaining a biological sample from the patient; measuring gene expression of the sample; and using the results obtained thereby to generate the report. The report can also contain an assessment of patient outcome and/or probability of risk relative to the patient population.

The present invention encompasses compositions containing at least one microarray probe set or PCR primer/probe set corresponding to Markers.

Articles of this invention include representations of the gene expression profiles useful for treating, diagnosing, prognosticating, and otherwise assessing diseases along with human or machine-readable instructions for their use. These profile representations are reduced to a medium that can be automatically read by a machine such as computer readable media (magnetic, optical, and the like). For example, the articles may comprise a CD ROM having computer instructions for comparing gene expression profiles of the portfolios of genes described above. The articles may also have gene expression profiles digitally recorded therein so that they may be compared with gene expression data from patient samples. Alternatively, the profiles can be recorded in different representational format. A graphical recordation is one such format. Clustering algorithms such as those incorporated in “DISCOVERY” and “INFER” software from Partek, Inc. mentioned above can best assist in the visualization of such data.

Different types of articles of manufacture according to the invention are media or formatted assays used to reveal gene expression profiles. These can comprise, for example, microarrays in which sequence complements or probes are affixed to a matrix to which the sequences indicative of the genes of interest combine creating a readable determinant of their presence. Alternatively, articles according to the invention can be fashioned into reagent kits for conducting hybridization, amplification, and signal generation indicative of the level of expression of the genes of interest for detecting cancer.

The present invention also encompasses kits for conducting an assay to determine lymph node metastasis from gastric carcinoma. Kits made according to the invention include formatted assays. These can include all or some of the materials needed to conduct the assays such as reagents and instructions and a medium through which nucleic acid sequences, their complements, or portions thereof are assayed. Where the assay is to be conducted in another format, the kit will have components peculiar to that format. For example, if the format is an amplification format such as PCR, Rolling Circle Amplification methods (RCA), Ligase Chain Reaction methods (LCR), Strand Displacement Amplification methods (SDA), Nucleic Acid Sequence Based Amplification methods (NASBA), then the kit will contain reagents and materials appropriate to such technologies.

In one embodiment, the kit includes reagents for amplifying and detecting Markers. Optionally, the kit includes sample preparation reagents and or articles (e.g., tubes) to extract nucleic acids from tissue such as lymph node tissue. The kits may also include articles to minimize the risk of sample contamination (e.g., disposable scalpel and surface for lymph node dissection and preparation).

In a preferred kit, reagents necessary for a one-tube QRT-PCR process are included such as reverse transcriptase, a reverse transcriptase primer, a corresponding PCR primer set (preferably for Markers and controls), a thermostable DNA polymerase, such as Taq polymerase, and a suitable detection reagent(s), such as, without limitation, a scorpion probe, a probe for a fluorescent 5′nuclease assay, a molecular beacon probe, a single dye primer or a fluorescent dye specific to double-stranded DNA, such as ethidium bromide. The primers are preferably in quantities that yield high concentrations. Thermostable DNA polymerases are commonly and commercially available from a variety of manufacturers. Additional materials in the kit may include: suitable reaction tubes or vials, a barrier composition such as a wax bead, optionally including magnesium; reaction mixtures (typically I OX) for the reverse transcriptase and the PCR stages, including necessary buffers and reagents such as dNTPs; nuclease—or RNase-free water;

RNase inhibitor; control nucleic acid (s) and/or any additional buffers, compounds, co-factors, ionic constituents, proteins and enzymes, polymers, and the like that may be used in reverse transcriptase and/or PCR stages of QRT-PCR reactions. Optionally, the kits include nucleic acid extraction reagents and materials.

It is also possible to assay the expression products of the gene Markers (themselves included in the definition of a Marker) such as polypeptides and proteins. A useful method for such an assay involves contacting a sample with antibodies directed to the Markers. These antibodies are useful in the development of very specific assays for the detection of Markers, and allow the tests to be carried out in many different formats. Preferably, the antibodies are labeled monoclonal antibodies. If such a Marker is a secreted protein or polypeptide then expressed between 100 and 1000 times in diseased tissues compared with its normal expression levels will be found. Kits for such assays include reagents typical for such assay formats such as labeled antibody reagents.

The following examples are provided to illustrate but not limit the claimed invention. All references cited herein are hereby incorporated by reference herein.

### EXAMPLE 1

### Marker Identification

Freshly frozen samples of 10 gastric carcinomas, 10 normal stomach tissues and 6 histologically confirmed normal lymph nodes were used to identify gene Markers for detection of lymph node metastasis from gastric carcinoma. RNAs were prepared from these tissues with RNeasy Mini Kit (Qiagen, Catalog# 74106) following standard vendor suggested protocols. Biotin labeled aRNA targets were prepared by RNA linear amplification (Ambion, MessageAmp II aRNA Amplification Kit, Catalog# 1751). Prepared targets were applied to Affymetrix HG U133A chips which were processed following standard Affymetrix suggested protocols. Chips results were collected and globally scaled to Target Signal of 600. Hierarchical clustering of microarray results (HG U I 33A chip set) was conducted and the chip results were statistically analyzed using Partek Pro 6.0. ANOVA and fold change analysis were applied to identify genes of interest. Genes with the following characteristics were selected as candidate markers for detection of lymph node metastasis from gastric carcinoma: a) expressed stably in normal stomach tissues, b) expression is maintained stably in gastric carcinomas, and c) there is a clear distinction in expression levels between the normal lymph node and gastric carcinoma.

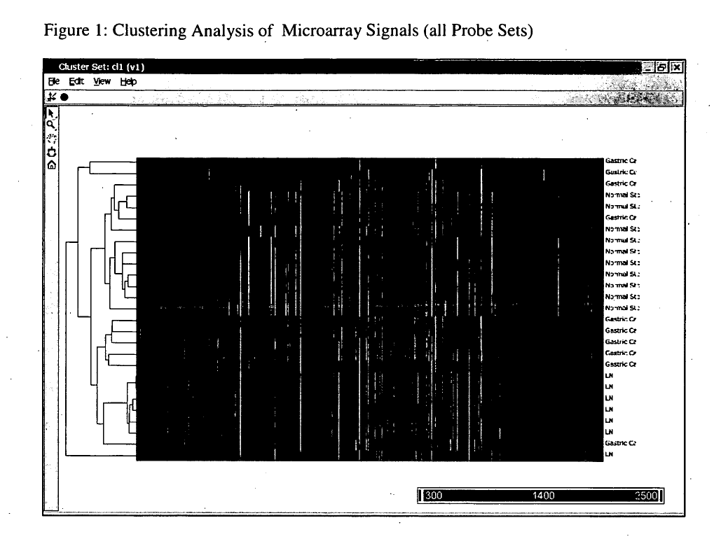

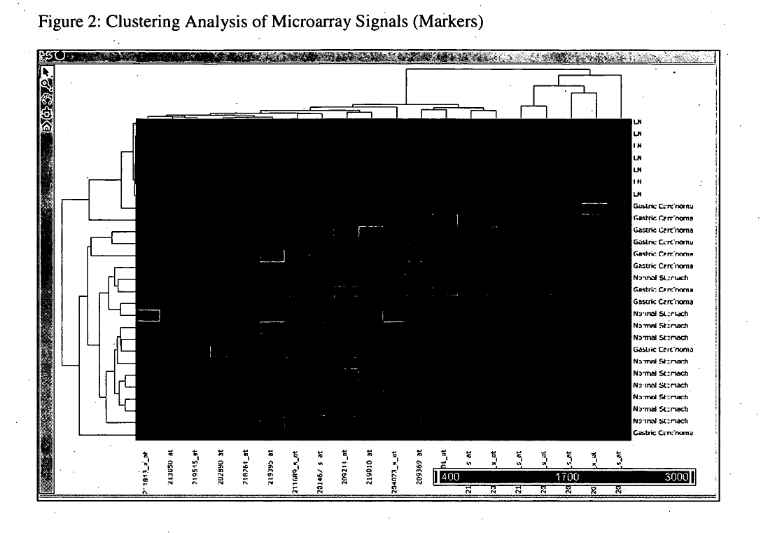

Twenty probe sets (representing 18 genes) met the criteria described above. The distinctive difference in gene signal levels between the normal lymph node group and the gastric carcinoma/normal stomach tissues was statistically significant. The microarray results are summarized in Table 1, average signal and standard deviation of each of the 20 probe sets are shown. More information about the Markers are shown in Table 2 and the clustering analysis is shown in FIG. 1 (all probe sets of HG U133A) and FIG. 2 (markers only). The expression of these Markers is increased in lymph nodes in the case of metastasis from gastric carcinoma. The markers are classified by functions as shown in Table 3.

TABLE 1
Summary of microarray results

| Affymetrix: | | Average | | | Standard Deviation | | |
|---|---|---|---|---|---|---|---|
| PSID | Genes | Lymph Node | Normal Stomach | Gastric Carcinoma | Lymph Node | Normal Stomach | Gastric Carcinoma |
| 209369_at | ANXA3 | 150 | 2077 | 1886 | 28 | 645 | 1193 |
| 218261_at | AP1M2 | 55 | 1061 | 735 | 25 | 237 | 446 |
| 201243_s_at | ATP1B1 | 431 | 10762 | 6659 | 76 | 2585 | 3075 |
| 204073_s_at | c11orf9 | 51 | 2642 | 1003 | 30 | 1099 | 630 |
| 219010_at | c1orf106 | 127 | 1780 | 1856 | 28 | 393 | 986 |
| 201131_s_at | CDH1 | 587 | 7333 | 6444 | 254 | 1054 | 4321 |
| 213050_at | COBL | 26 | 709 | 317 | 6 | 172 | 213 |
| 211843_x_at | CYP3A7 | 18 | 788 | 219 | 4 | 349 | 128 |
| 217901_at | DSG2 | 189 | 3077 | 2162 | 113 | 920 | 1445 |
| 210827_s_at | ELF3 | 183 | 2498 | 2282 | 50 | 315 | 1456 |
| 202454_s_at | ERBB3 | 124 | 2649 | 2411 | 32 | 489 | 1472 |
| 219545_at | KCTD14 | 20 | 727 | 362 | 2 | 184 | 201 |
| 209211_at | KLF5 | 52 | 1227 | 1312 | 23 | 266 | 852 |
| 209008_x_at | KRT8 | 158 | 6815 | 7412 | 20 | 1736 | 4803 |
| 202890_at | MAP7 | 31 | 921 | 756 | 22 | 226 | 335 |
| 201468_s_at | NQO1 | 225 | 7017 | 3107 | 57 | 1242 | 1939 |
| 210519_s_at | NQO1 | 235 | 5276 | 3688 | 51 | 1126 | 2123 |
| 201467_s_at | NQO1 | 15 | 1585 | 1076 | 7 | 293 | 678 |
| 219395_at | RBM35B | 78 | 1145 | 867 | 46 | 337 | 530 |
| 211689_s_at | TMPRSS2 | 9 | 1549 | 632 | 5 | 569 | 432 |

TABLE 2
Gene Markers and Their Affymetrix Probe Set Information

| | | | | |
|---|---|---|---|---|
| | | Affymetrix | NCBI Reference | |
| Gene | Gene Name/Description | PSID | Sequences (mRNA) | Seq. ID No. |

| | | | |
|---|---|---|---|
| ANXA3 | annexin A3 | 209369_at | NM_005139 |
| AP1M2 | adaptor-related protein | 218261_at | NM_005498 |
| | complex 1, mu 2 subunit | | |
| ATP1B1 | ATPase, Na+/K+ transporting, | 201243_s_at | NM_001677, |
| | beta 1 polypeptide | | NM_001001787 |
| C11orf9 | chromosome 11 open reading | 204073_s_at | NM_013279 |
| | frame 9 | | |
| C1orf106 | Hypethetical Protein FLJ10901, | 219010_at | NM_018265 |
| | chromosome 1 open reading | | |
| | frame 106 | | |
| CDH1 | cadherin 1, type 1, E-cadherin | 201131_s_at | NM_004360 |
| | (epithelial) | | |
| COBL | cordon-bleu homolog (mouse) | 213050_at | NM_015198 |
| CYP3A7 | cytochrome P450, family 3, | 211843_x_at | NM_000765 |
| | subfamily A, polypeptide 7 | | |
| DSG2 | desmoglein 2 | 217901_at | NM_001943 |
| ELF3 | E74-like factor 3 (ets domain | 210827_s_at | NM_004433 |
| | transcription factor, epithelial- | | |
| | specific) | | |
| ERBB3 | v-erb-b2 erythroblastic | 202454_s_at | NM_001982, |
| | leukemia viral oncogene | | NM_001005915 |
| | homolog 3 (avian) | | |
| KCTD14 | potassium channel | 219545_at | NM_023930 |
| | tetramerisation domain | | |
| | containing 14 | | |
| KLF5 | Kruppel-like factor 5 (intestinal) | 209211_at | NM_001730 |
| KRT8 | keratin 8 | 209008_x_at | NM_002273 |
| MAP7 | microtubule-associated protein 7 | 202890_at | NM_003980 |
| NQO1 | NAD(P)H dehydrogenase, | 201468_s_at | NM_000903 |
| | quinone 1 | | NM_001025433 |
| | | | NM_001025434 |
| NQO1 | NAD(P)H dehydrogenase, | 210519_s_at | NM_000903 |
| | quinone 1 | | NM_001025433 |
| | | | NM_001025434 |
| NQO1 | NAD(P)H dehydrogenase, | 201467_s_at | NM_000903, |
| | quinone 1 | | NM_001025433, |
| | | | NM_001025434 |
| RBM35B | hypothetical protein FLJ21918 | 219395_at | NM_024939 |
| TMPRSS2 | transmembrane protease, | 211689_s_at | NM_005656 |
| | serine 2 | | |

TABLE 3
Markers Classified by Functions

| Function | Number | Genes |
|---|---|---|
| Signal Transduction | 5 | ANXA3, ATP1B1, AP1M2, KCTD14, |
| | | ERBB3, |
| Transcription Factor | 2 | ELF3, KLF5 |
| Intercellular Junction | 2 | CDH1, DSG2, |
| Unknown | 4 | RBM35B (FLJ21918), COBL, |
| | | C1orf106 (FLJ10901), C11orf9 |
| Cell Structure | 2 | KRT8, MAP7 |
| Cytoplasmic Enzyme | 3 | NQO1, CYP3A7, TMPRSS2 |
| Total | 18 | |

### EXAMPLE 2

### Confirmation Study (1)—Microarray Test

Confirmation studies were performed to validate the Markers selected in Example 1. These markers were examined to determine whether their expression is maintained in lymph nodes with metastasis from gastric carcinomas of various differentiation statuses. Affymetrix DNA microarrays (HG U 133A) were used in this study. Tissue of 6 normal lymph nodes and 3 lymph nodes with confirmed metastasis from gastric carcinoma were tested. RNAs were prepared from these samples as described above. Biotin-labeled aRNA targets were prepared as in Example 1. Targets were applied to Affymetrix HG U133A chips. Results from the chips were scaled to Target Signal of 600 and analyzed.

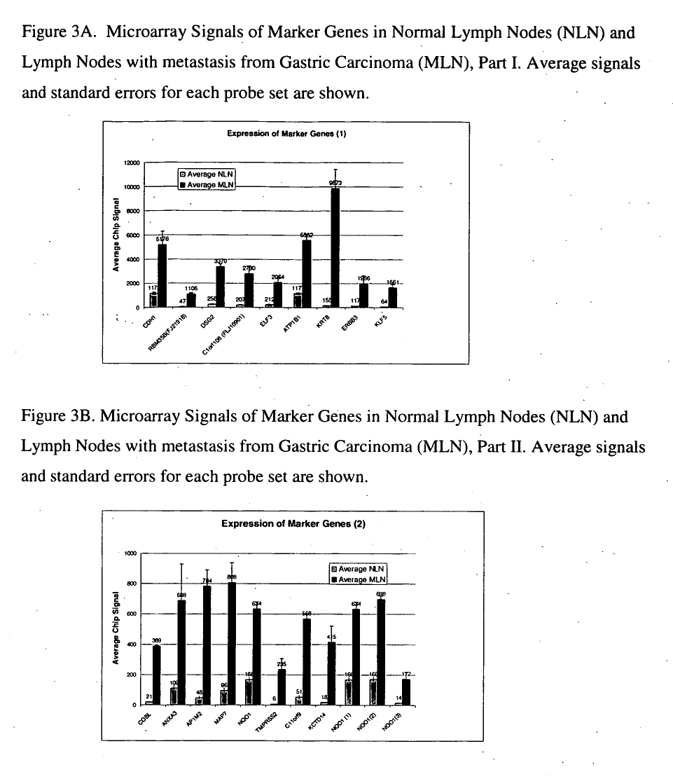

The average signal of each probe set and standard error are plotted in FIG. 3A, FIG. 3B.

### EXAMPLE 3

### Confirmation Study (2)—Realtime RT-PCR Test

Studies were conducted to validate the performance of selected gene markers using real time RT-PCR. 10 normal lymph nodes and 16 lymph nodes with histologically confirmed metastasis from gastric carcinoma were tested (including all the samples tested with DNA microarray in Confirmation Study 1). The 10 histologically confirmed normal lymph nodes were from 7 different patients. The 16 lymph nodes with confirmed metastasis from gastric carcinoma were from 13 different patients. The malignancy statuses of the original carcinoma that metastasized to lymph nodes are: 3 well differentiated (contributed to 6 lymph nodes), 7 poorly differentiated (contributed to 7 nodes), 3 moderately differentiated (contributed to 3 lymph nodes).

All RNAs were prepared as described above. cDNAs were first prepared from 5 μg RNA in 100 μL reaction volume with a High Capacity cDNA Archive Kit (ABI) following the vendor suggested protocol. 2 μL of the cDNA that was synthesized was used in the real time, PCR reaction (RT-PCR) in a volume of 50 μL. One cDNA was prepared from each RNA sample. TaqMan Gene Expression Assays were purchased from Applied Biosystems Inc (ABI) for each of the gene markers. RT-PCR tests were performed using these assays with TaqMan Universal PCR Master Mix without AmpErase UNG (ABI) under vendor suggested conditions, one reaction (assay) in each tube. Duplicate or triplicate PCR reactions were performed on each cDNA generated. PCR was run on an ABI 7900 HT analyzer.

Assay ID and Context sequences for each assay (one primer/probe set for a gene) are shown in Table 4:

TABLE 4
TaqMan Gene Expression Assay (Applied Biosystems, Inc)

| | | | | |
|---|---|---|---|---|
| | | NCBI | | |
| | | Reference | | Seq. ID of |
| | | Seqences | | Context |
| Gene | ABI Assay ID No. | (mRNA) | Context Sequence | Seq. |

| | | | |
|---|---|---|---|
| ANXA3 | Hs00192983_m1 | NM_005139 | TCAGAGGAATTGGAACTGATGAGAA |
| AP1M2 | HS00194014_m1 | NM_005498 | AGGGCAAGCCATTGATCAGCCGCAA |
| ATP1B1 | Hs00426868_g1 | NM_001677, | CAGTGCACTGGCAAGCGAGATGAAG |
| | | NM_001001787 | |
| C11orf9 | Hs00973712_g | NM_013279 | GTCATGGCCTTCAGCGTGGTGTCCA |
| C1orf106 | Hs00250963_m1 | NM_018265 | TGGGACAGCTGGGGAAGCTCCAGGC |
| CDH1 | Hs00170423_m1 | NM_004360 | CCCGCCCCATCAGGCCTCCGTTTCT |
| COBL | Hs00323596_m1 | NM_015198 | CTGAGAAAGGTGCCCTTGCTCGTGT |
| CYP3A7 | Hs02511627_s1 | NM_000765 | TTATTTTGATGGCTCTAACAGTGAC |
| DSG2 | Hs00170071_m1 | NM_001943 | CAGAGAGGAACACAGCAGCTACACT |
| ELF3 | Hs00231786_m1 | NM_004433 | CAAGCACGCGCCCAGAGGCACCCAC |
| ERBB3 | Hs00176538_m1 | NM_001982, | GCAACTCTCAGGCAGTGTGTCCTGG |
| | | NM_001005915 | |
| KCTD14 | Hs01928822_s1 | NM_023930 | Not available from Vendor |
| KLF5 | Hs00156145_m1 | NM_001730 | ACTGCGATTACCCTGGTTGCACAAA |
| KRT8 | Hs01595539_g1 | NM_002273 | Not available from Vendor |
| MAP7 | Hs00187725_m1 | NM_003980 | GCGAAGCGAAACAGCACCCGACAGC |
| NQO1 | Hs00168547_m1 | NM_000903 | GAGCCATGGTCGGCAGAAGAGCACT |
| | | NM_001025433 | |
| | | NM_001025434 | |
| RBM35B | Hs00227840_m1 | NM_024939 | GTGCTGCAGCAGTTCTCACAGCTGG |
| TMPRSS2 | Hs00237175_m1 | NM_005656 | TCGGTGTGTTCGCCTCTACGGACCA |
| HPRT1* | 185278564 | NM_000194 | Not available from Vendor |

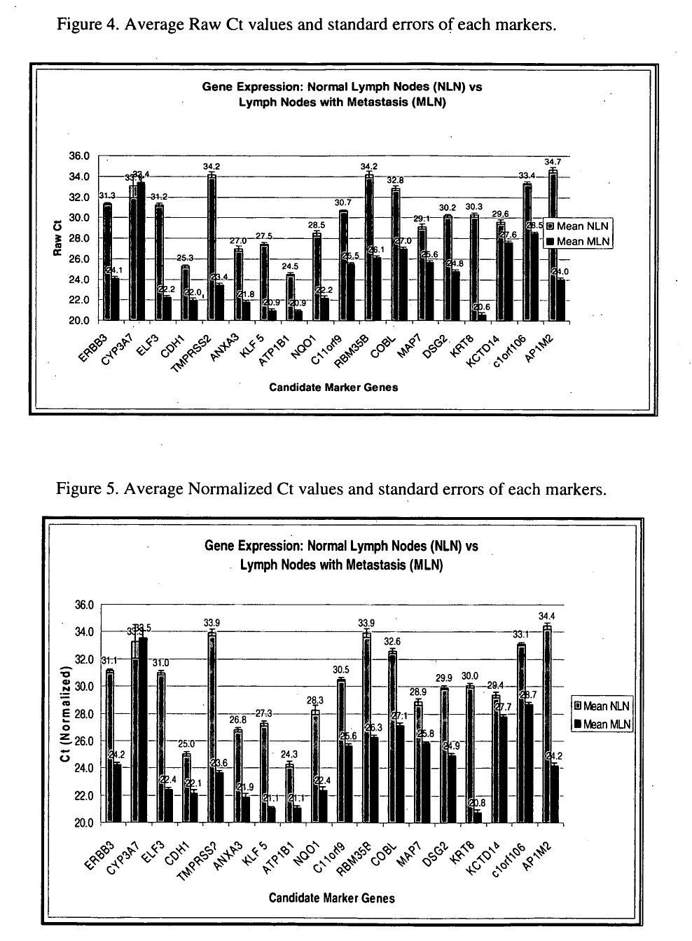

Ct values were collected for each of the marker genes for samples from normal lymph nodes (NLN) and lymph nodes with confirmed metastasis from gastric carcinoma (MLN). Raw Ct values for each gene were normalized against the Ct of the house keeping gene, HPRT 1. Both the raw Ct and the normalized Ct values across multiple RT-PCR Runs were summarized and analyzed. The average raw Ct and standard error of each gene for both sample groups (NLN, MLN) were calculated as shown in FIG. 4. The average normalized Ct and standard error of each marker gene for both sample groups (NLN, MLN) were calculated as shown in FIG. 5. For the gene CYP3A7, Ct values were undetermined in 16 out of 26 PCR reactions in NLN group, and 2 out of 35 PCR reactions on MLN group, while typical Ct values of the housekeeping (consitutively expressed) gene HPRT1 were obtained from the same cDNA. The PCR detection rates for gene CYP3A7 are significantly different between NLN and MLN groups (P<0.0001). For CYP3A7 , only determined Ct values were used in the calculation of average, standard deviation, standard error and ANOVA. ANOVA was performed on raw and normalized Ct values to test the significance of difference between the two groups (NLN, MLN), as shown in Table 5 and Table 6. There is no case of undetermined Ct value in PCR reactions on genes tested other than CYP3A7.

TABLE 5
Data Summary and ANOVA Results on raw Ct values

| | | Mean | Std | Min | Max | Mean | Std | Min | Max | MS | MS |
|---|---|---|---|---|---|---|---|---|---|---|---|
| Genes | p-value | (MLN) | (MLN) | (MLN) | (MLN) | (NLN) | (NLN) | (NLN) | (NLN) | (model) | (Error) |
| ELF3 | 9.4E−43 | 22.2 | 0.9 | 20.7 | 23.8 | 31.2 | 0.9 | 28.3 | 32.0 | 1204 | 0.9 |
| AP1M2 | 1.8E−42 | 24.0 | 1.0 | 22.0 | 25.8 | 34.7 | 1.3 | 31.1 | 36.4 | 1689 | 1.2 |
| ERBB3 | 6.6E−41 | 24.1 | 1.0 | 22.7 | 25.9 | 31.3 | 0.5 | 30.4 | 32.5 | 791 | 0.7 |
| TMPRSS2 | 1.1E−39 | 23.4 | 1.0 | 21.9 | 25.7 | 34.2 | 1.6 | 30.3 | 36.9 | 1713 | 1.6 |
| KRT8 | 5.5E−36 | 20.6 | 1.5 | 18.0 | 24.1 | 30.3 | 1.1 | 27.7 | 31.9 | 1406 | 1.8 |
| C11orf9 | 2.4E−32 | 25.5 | 0.8 | 24.3 | 27.0 | 30.7 | 0.4 | 29.7 | 31.0 | 329 | 0.4 |
| RBM35B(FLJ21918) | 3.8E−31 | 26.1 | 1.1 | 24.6 | 28.9 | 34.2 | 1.6 | 30.7 | 38.0 | 971 | 1.8 |
| DSG2 | 2.4E−30 | 24.8 | 1.1 | 22.5 | 27.1 | 30.2 | 0.6 | 28.9 | 31.3 | 435 | 0.9 |
| C1orf106(FLJ10901) | 1.4E−29 | 28.5 | 1.0 | 26.6 | 30.5 | 33.4 | 0.8 | 31.9 | 34.5 | 355 | 0.8 |
| KLF 5 | 2.6E−29 | 20.9 | 1.1 | 19.6 | 23.5 | 27.5 | 0.7 | 25.8 | 28.3 | 520 | 0.9 |
| COBL | 4.5E−28 | 27.0 | 1.0 | 24.7 | 28.9 | 32.8 | 1.3 | 29.9 | 34.9 | 516 | 1.3 |
| NQO1 | 3.5E−26 | 22.2 | 1.3 | 19.0 | 23.7 | 28.5 | 1.4 | 26.6 | 30.8 | 592 | 1.7 |
| ATP1B1 | 7.7E−26 | 20.9 | 0.8 | 19.1 | 22.3 | 24.5 | 0.7 | 23.6 | 25.8 | 195 | 0.6 |
| ANXA3 | 5.2E−22 | 21.8 | 1.0 | 20.4 | 24.6 | 27.0 | 1.2 | 24.6 | 28.8 | 332 | 1.2 |
| MAP7 | 2.8E−17 | 25.6 | 1.0 | 24.2 | 27.2 | 29.1 | 1.3 | 26.6 | 30.6 | 180 | 1.3 |
| CDH1 | 9.8E−16 | 22.0 | 1.4 | 19.8 | 26.3 | 25.3 | 0.8 | 24.5 | 28.5 | 164 | 1.4 |
| KCTD14 | 1.4E−09 | 27.6 | 1.0 | 25.5 | 29.1 | 29.6 | 1.2 | 26.7 | 31.5 | 62 | 1.2 |
| CYP3A7* | 7.1E−01 | 33.4 | 2.6 | 30.6 | 38.7 | 33.1 | 3.6 | 29.5 | 39.6 | 1 | 7.9 |

TABLE 6
Data Summary and ANOVA Results on Normalized Ct values

| | | Mean | Std | Min | Max | Mean | Std | Min | Max | MS | MS |
|---|---|---|---|---|---|---|---|---|---|---|---|
| Genes | p-value | (MLN) | (MLN) | (MLN) | (MLN) | (NLN) | (NLN) | (NLN) | (NLN) | (Model) | (Error) |
| AP1M2 | 5.2E−42 | 24.2 | 1.0 | 22.2 | 25.9 | 34.4 | 1.2 | 31.5 | 37.0 | 1559 | 1.2 |
| ELF3 | 1.5E−40 | 22.4 | 1.1 | 21.2 | 24.5 | 31.0 | 0.8 | 28.7 | 32.5 | 1095 | 0.9 |
| TMPRSS2 | 7.4E−40 | 23.6 | 1.2 | 21.9 | 25.8 | 33.9 | 1.2 | 30.8 | 36.1 | 1582 | 1.4 |
| KRT8 | 7.3E−39 | 20.8 | 1.3 | 18.2 | 23.6 | 30.0 | 0.9 | 28.1 | 31.1 | 1288 | 1.3 |
| ERBB3 | 1.0E−38 | 24.2 | 0.9 | 22.8 | 25.7 | 31.1 | 0.8 | 30.2 | 33.0 | 703 | 0.7 |
| RBM35B(FLJ21918) | 5.4E−33 | 26.3 | 0.7 | 25.4 | 28.4 | 33.9 | 1.6 | 31.2 | 38.3 | 874 | 1.4 |
| KLF 5 | 1.7E−31 | 21.1 | 0.8 | 19.6 | 22.9 | 27.3 | 0.8 | 26.2 | 29.3 | 475 | 0.6 |
| C1orf106(FLJ10901) | 2.1E−31 | 28.7 | 0.8 | 27.1 | 30.2 | 33.1 | 0.6 | 32.2 | 34.5 | 297 | 0.5 |
| DSG2 | 7.5E−31 | 24.9 | 1.0 | 22.5 | 26.6 | 29.9 | 0.6 | 28.7 | 30.8 | 370 | 0.7 |
| COBL | 9.7E−29 | 27.1 | 1.0 | 25.4 | 28.5 | 32.6 | 1.0 | 30.3 | 34.4 | 445 | 1.0 |
| C11orf9 | 6.9E−26 | 25.6 | 0.9 | 23.9 | 27.3 | 30.5 | 0.6 | 29.7 | 31.8 | 293 | 0.7 |
| ANXA3 | 1.0E−20 | 21.9 | 1.3 | 20.1 | 25.6 | 26.8 | 0.7 | 25.6 | 27.8 | 296 | 1.2 |
| NQO1 | 1.7E−20 | 22.4 | 1.5 | 19.1 | 24.7 | 28.3 | 1.7 | 26.9 | 31.8 | 517 | 2.6 |
| ATP1B1 | 4.9E−18 | 21.1 | 1.0 | 19.1 | 22.4 | 24.3 | 1.0 | 23.1 | 26.7 | 153 | 1.0 |
| MAP7 | 3.5E−17 | 25.8 | 0.9 | 24.1 | 27.7 | 28.9 | 1.1 | 25.9 | 30.0 | 139 | 1.0 |
| CDH1 | 5.8E−13 | 22.1 | 1.5 | 20.1 | 26.2 | 25.0 | 0.7 | 24.2 | 28.0 | 125 | 1.5 |
| KCTD14 | 5.5E−07 | 27.7 | 1.1 | 25.1 | 29.5 | 29.4 | 1.1 | 25.7 | 30.8 | 39 | 1.2 |
| CYP3A7* | 0.82 | 33.5 | 3.0 | 29.9 | 39.7 | 33.3 | 3.9 | 28.5 | 40.0 | 0.5 | 10.2 |

Except for CYP3A7 (undetermined Ct observed in many PCR reactions), significantly increased expression of the 17 Markers were observed in all of the 16 lymph nodes with metastasis from gastric carcinoma of various differentiation states (from 13 patients). The background expression levels of these Marker genes are consistent among the 10 normal lymph nodes tested, which are significantly lower than that of lymph nodes with metastasis from gastric carcinoma.

Receiver Operating Characteristic (ROC) curves were generated for each of the 17 Markers (excluding CYP3A7) to show the potential of them to be used in discriminative assays against the two sample groups (MLN, NLN). A summary of the analysis of these curves is shown in Table 7. AUC (Area Under Curve) for each of the gene markers was calculated. Two Cut-off Cts were tested, and the respective sensitivity and specificity for each Cut-off Ct value are shown in Table7.

TABLE 7
Summary of ROC Curve for each Gene Marker Ct results

| | | Situation A | | | Situation B | | |
|---|---|---|---|---|---|---|---|
| Gene | ROC-AUC | Cutoff Ct #1 | Sensitivity #1 | Specificity #1 | Cutoff Ct #2 | Sensitivity #2 | Specificity #2 |
| DSG2 | 1 | 28.026 | 1.00 | 1.00 | 29.48 | 1.00 | 0.89 |
| RBM35B | 1 | 29.82 | 1.00 | 1.00 | 32.64 | 1.00 | 0.89 |
| COBL | 1 | 29.40 | 1.00 | 1.00 | 31.27 | 1.00 | 0.89 |
| ELF3 | 1 | 26.06 | 1.00 | 1.00 | 30.71 | 1.00 | 0.89 |
| AP1M2 | 1 | 28.41 | 1.00 | 1.00 | 33.63 | 1.00 | 0.89 |
| NQO1 | 1 | 25.17 | 1.00 | 1.00 | 27.08 | 1.00 | 0.89 |
| C1orf106 | 1 | 31.20 | 1.00 | 1.00 | 32.17 | 1.00 | 0.89 |
| ATP1B1 | 1 | 22.90 | 1.00 | 1.00 | 23.66 | 1.00 | 0.89 |
| ERBB3 | 1 | 28.16 | 1.00 | 1.00 | 30.62 | 1.00 | 0.89 |
| C11orf9 | 1 | 28.35 | 1.00 | 1.00 | 30.05 | 1.00 | 0.90 |
| KLF5 | 1 | 24.65 | 1.00 | 1.00 | 26.61 | 1.00 | 0.90 |
| KRT8 | 1 | 25.90 | 1.00 | 1.00 | 29.035 | 1.00 | 0.89 |
| TMPRSS2 | 1 | 27.98 | 1.00 | 1.00 | 32.70 | 1.00 | 0.89 |
| ANXA3 | 0.998 | 24.40 | 0.97 | 1.00 | 25.30 | 1.00 | 0.90 |
| MAP7 | 0.974 | 26.545 | 0.714 | 1.00 | 27.03 | 0.97 | 0.89 |
| CDH1 | 0.945 | 23.83 | 0.94 | 1.00 | 24.69 | 0.94 | 0.89 |
| KCTD14 | 0.898 | 26.52 | 0.17 | 1.00 | 28.30 | 0.69 | 0.89 |

### EXAMPLE 4

### Antibodies (Prophetic)

C-terminal Markers-derived peptides are synthesized, coupled to keyhole limpet hemocyanin, and used to immunize rabbits for production of polyclonal antibodies. The sera are tested for reactivity against the corresponding peptide with ELISA, and the positive batches are affinity-purified. The purified antibody specifically detects the protein that has the peptide epitope in tissue sections. This is verified by complete abolishment of the signal if the corresponding peptide is added simultaneously with the antibody. In addition to this polyclonal antibody, which works well in immunohistochemistry, monoclonal antibodies able to detect the protein in its natural fold are produced. To produce monoclonal antibodies, a purified antigen, produced in mammalian cells to ensure natural fold and posttranslational modifications, are generated. The antigen, Markers-IgG constant part fusion protein, is expressed in mouse myeloma cells, and the secreted protein is purified using the Fc part as bait. This purified antigen is recognized in Western blot by the C-terminal polyclonal antibody, and by other anti-Markers peptide antibodies. The antigen is used to generate mouse monoclonal antibodies against Markers by selecting out of the positive clones those that produced antibodies that react against Markers instead of the IgG constant part.

Kits for the clinical identification of Markers can be readily fashioned employing these and similar antibodies. Such kits would include antibodies directed to Markers identification, appropriate indicator reagents (e.g., enzymes, labels, and the like), and (optionally) other reagents useful in the clinical application of such a kit such as dilution buffers, stabilizers, and other materials typically used in such assays. The kits would be used to detect Markers in body fluids to screen or follow-up Markers expressing cancers, and to screen the presence of Markers protein in tissue samples. Kits that include antibodies as described are also made to conduct immunocytochemistry assays for the tissue of interest. These kits include positive staining for the Marker protein to indicate metastasis.

### EXAMPLE 5

### Enzyme Immunoassay (Prophetic)

Immunoassays are prepared for the Markers. To develop an Enzyme Immunoassay (EIA) procedure, antigen standards comprising a digest of stomach tumor specimens (shown to contain the antigen by immunoperoxidase staining) are used. Human primary stomach cancer specimens are pooled and homogenized in 10 volumes of 10 mM Tris buffer, pH 7.4, containing 0.2% (w/v) sodium deoxycholate at 4 C. The homogenate is quickly brought to 37 C and the following reagents (final concentration) are added while stirring: 1 mM cysteine (Sigma), 1 mM EDTA (Sigma), and papain (0.8 unit/ml) (Boehringer-Mannheim, Indianapolis, Ind.). After 5 minutes, digestion is stopped by the addition of 5 mM iodoacetamide (Sigma). The homogenate is centrifuged at 100,000×g for 1 hour at 4 C, then extensively dialyzed against 10 mM Tris/0.9% NaCl solution buffer, pH 7.4, containing phenylmethysulfonyl fluoride and aminocaproic acid, each at 10 mM. The homogenate is frozen in small aliquots at a concentration of 0.5 mg of protein/mi.

The dose response curve that will be generated for the immunoassay procedure measuring Markers will demonstrate linearity at clinically significant low levels of concentration.

Solid-phase preparations of the antibodies described in Example 6 are prepared using CNBr-activated Sepharose (Pharmacia). Microtiter plates (Nunc I Immunoplates; Grand Island Biological Co., Grand Island, N.Y.) are coated with the antibodies (200 μl/well) in 50 mM carbonate-bicarbonate buffer, pH 9.6, for 18 hours at 4 C. After removal of the antibody solution, residual protein binding sites on the plastic are blocked by the addition of 200 μl of assay buffer [PBS containing 1% (v/v) rabbit serum and 1% (w/v) bovine albumin]. After 1 hour of incubation at room temperature, the coated plates are used immediately for the assay procedure.

To perform the assay, samples, diluted in assay buffer, are applied for 1-5 hours at 37 C. After 3 washes using assay buffer, 200 μl of the antibody covalently conjugated to horseradish peroxidase (Sigma, Type VI) is applied to each well for 1.5 hours at 37 C. The conjugate is diluted to a concentration of 0.5 μg of immunoglobulin per ml of PBS containing 10% (v/v) murine serum. Following a wash procedure as above, 200 μl of substrate per well are applied for 0.5 hours at room temperature. Substrate solution contains 0.4 mg of o-phenylenediamine per ml of pH 5.0 citrate buffer and 0.003% hydrogen peroxide. The reaction is stopped by addition of 50 μl of 2N sulfuric acid, and absorbance is monitored at 488 nM using an enzyme assay plate reader (Fisher Scientific Co., Pittsburgh, Pa.).

The percentage of bound enzyme conjugate is calculated by the formula:
(B−B₀)(Bₜ−B₀)(100)
where B=absorbance of the sample, Bₜ=maximal absorbance, and B₀=absorbance of the blank. Each assay is performed in triplicate using a standard digest and 26-fold diluted serum samples diluted in assay buffer. Specificity of the immunoassay is examined by substituting various antibody reagents at the solid phase, including an antibody to CEA and nonimmune rabbit serum.

Samples obtained from patients with cancers corresponding to those described above are also evaluated. The incidence of elevated Markers values is 90%. Mean values from the group with cancer are significantly higher than control levels (about 250% higher).

Using a limited number of postoperative stomach cancer patients with primary localized disease, a significant decrease in Markers occurs. These data indicate a relationship between Markers levels and tumor load. Such measurements are thus valuable for patient monitoring.

### REFERENCES

Ajisaka, H. and K. Miwa (2003). “Micrometastases in sentinel nodes of gastric cancer.” Br J Cancer 89(4): 676-80.
Chausovsky, G., M. Luchansky, et al. (1999). “Expression of cytokeratin 20 in the blood of patients with disseminated carcinoma of the pancreas, colon, stomach, and lung.” Cancer 86(11): 2398-405.
Chen, X. M., G. Y. Chen, et al. (2004). “Detection of micrometastasis of gastric carcinoma in peripheral blood circulation.” World J Gastroenterol 10(6): 804-8.
Hartgrink, H. H. and C. J. van de Velde (2005). “Status of extended lymph node dissection: locoregional control is the only way to survive gastric cancer.” J Surg Oncol 90(3): 153-65.
Heeren, P. A., W. Kelder, et al. (2005). “Prognostic value of nodal micrometastases in patients with cancer of the gastro-oesophageal junction.” Eur J Surg Oncol 31(3): 270-6.
Jemal, A., T. Murray, et al. (2005). “Cancer statistics, 2005.” CA Cancer J Clin 55(1): 10-30.
Kim, T. M., H. J. Jeong, et al. (2005). “Determination of genes related to gastrointestinal tract origin cancer cells using a cDNA microarray.” Clin Cancer Res 11(1): 79-86.
Kitagawa, Y., H. Fujii, et al. (2005). “Recent advances in sentinel node navigation for gastric cancer: a paradigm shift of surgical management.” J Surg Oncol 90(3): 147-51; discussion 151-2.
Kumar, V., R. S. Cotran, et al. (2003). Basic Pathology. Philadelphia, Saunders.
Matsuda, J., Y. Kitagawa, et al. (2004). “Significance of metastasis detected by molecular techniques in sentinel nodes of patients with gastrointestinal cancer.” Ann Surg Oncol 11(3 Suppl): 250S-4S.
Okada, Y., Y. Fujiwara, et al. (2001). “Genetic detection of lymph node micrometastases in patients with gastric carcinoma by multiple-marker reverse transcriptase-polymerase chain reaction assay.” Cancer 92(8): 2056-64.
Parkin, D. M., F. Bray, et al. (2001). “Estimating the world cancer burden: Globocan 2000.” Int J Cancer 94(2): 153-6.
Parkin, D. M., F. I. Bray, et al. (2001). “Cancer burden in the year 2000. The global picture.” Eur J Cancer 37 Suppl 8: S4-66.
Shibuya, K., C. D. Mathers, et al. (2002). “Global and regional estimates of cancer mortality and incidence by site: II. Results for the global burden of disease 2000.” BMC Cancer 2: 37.
Songun, I., J. J. Bonenkamp, et al. (1996). “Prognostic value of resection-line involvement in patients undergoing curative resections for gastric cancer.” Eur J Cancer 32A(3): 433-7.

1. A method of diagnosing lymph node metastasis from gastric carcinoma comprising the steps:
a) obtaining a biological sample from a patient, and
b) measuring Marker levels in the sample;
wherein the levels above the pre-determined cut-off levels are indicative of lymph node metastasis from gastric carcinoma.

2. The method of claim 1 wherein the Markers are genes, gene fragments, or complements thereof.

3. The method of claim 1 wherein the Markers are proteins, protein fragments, or polypeptides.

4. The method of claim 1 further comprising the use of another diagnostic method.

5. The method of claim 4 wherein the other diagnostic method comprises conducting a serum assay.

6. The method of claim 1 further comprising the measurement of a consitutively expressed gene.

7. The method of claim 1 having a sensitivity of at least 80%.

8. The method of claim 1 having a specificity of at least 70%.

9. A method of determining or adjusting therapy for a gastric carcinoma patient comprising the steps:
a. obtaining a biological sample from a gastric cancer patient; and
b. measuring the levels in the sample of a Marker;
wherein levels above or below a cut-off levels are consistent with a course of disease or physical condition for which a therapy is determined or adjusted.

10. The method of claim 9 wherein the Marker is a gene, a gene fragment, or the complement thereof.

11. The method of claim 9 wherein the Marker is a protein, protein fragment, or polypeptide.

12. The method of claim 9 further comprising the measurement of a consitutively expressed gene.

13. The method of claim 9 wherein the specificity is at least 70%.

14. The method of claim 9 wherein the sensitivity is at least about 80%.

15. The method of claim 1 conducted with a microarray.

16. The method of claim 9 conducted with a microarray.

17. The method of claim 1 conducted by using an amplification process.

18. The method of claim 17 wherein the amplification process is a PCR.

19. The method of claim 18 wherein the PCR s an RT-PCR.

20. The method of claim 9 conducted by using an amplification process.

21. The method of claim 20 wherein the amplification process is a PCR.

22. The method of claim 21 wherein the PCR is an RT-PCR.

23. A kit for conducting an assay to determine gastric cancer diagnosis in a biological sample comprising materials for detecting a Marker.

24. The kit of claim 23 including a microarray and reagents.

25. The kit of claim 23 including PCR reagents.

26. The kit of claim 23 including instructions.
